SEQUENCE LISTING (73 sequences)

SEQ ID NO: 1 - DNA, 7801 bp, Homo sapiens
gtggtggccgcgcacccggccgcggctgattcattcacttcaagtgccgtgcagaaggctcggcaggcggggcgggcgtggggccgcggctccgggttggggaccgaggagatccggctgtggaccagacgctcctctgcggggcgggcacccaagcgcgctcgccaccccctcgccatccgctagagccgggctcctggactgggactcgggcccgccgcacagttgaaaagtcgcatagtggtttttccgctcgcgtcgctgtgtgaaagttggctcgccgctctttgcacgccctccctggaggccgacccgagacgccaagctggagagaccgtgcctccccgaggccggccgccccgcgagcacagcctccgcccccgttgcactgccgggctgggcaatatgaaggagcagccctcatgtgccggcaccgggcatccgagcatggcggggtatggcaggatggccccctttgaactcgctagcggacccgtgaagcgcttgagaactgagtccccctttccctgtctcttcgcagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 2 - protein, 585 aa, Homo sapiens
MKEQPSCAGTGHPSMAGYGRMAPFELASGPVKRLRTESPFPCLFAEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 3 - DNA, 21 bp, Artificial Sequence
  PDE4D1D2_forward primer
aatatgaaggagcagccctca

SEQ ID NO: 4 - DNA, 18 bp, Artificial Sequence
  PDE4D1D2_reverse primer
gtctcgctggccagtttc

SEQ ID NO: 5 - DNA, 18 bp, Artificial Sequence
  PDE4D1D2 probe
catccgagcatggcggga

SEQ ID NO: 6 - DNA, 7715 bp, Homo sapiens
gtggtggccgcgcacccggccgcggctgattcattcacttcaagtgccgtgcagaaggctcggcaggcggggcgggcgtggggccgcggctccgggttggggaccgaggagatccggctgtggaccagacgctcctctgcggggcgggcacccaagcgcgctcgccaccccctcgccatccgctagagccgggctcctggactgggactcgggcccgccgcacagttgaaaagtcgcatagtggtttttccgctcgcgtcgctgtgtgaaagttggctcgccgctctttgcacgccctccctggaggccgacccgagacgccaagctggagagaccgtgcctccccgaggccggccgccccgcgagcacagcctccgcccccgttgcactgccgggctgggcaatatgaaggagcagccctcatgtgccggcaccgggcatccgagcatggcgggaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 7 - protein, 507 aa, Homo sapiens
MASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 8 - DNA, 7783 bp, Homo sapiens
aatacttgttgcaataattgcccacgatagctgctcaaacaagagagttggaattcatctgtaaaaatcactacatgtaacgtaggagacaagaaaaatattaatgacagaagatctgcgaacatgatgcacgtgaataattttccctttagaaggcattcctggatatgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 9 - protein, 673 aa, Homo sapiens
MMHVNNFPFRRHSWICFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 10 - DNA, 8240 bp, Homo sapiens
cccctctcggtagccctgaggctctggcgccttcaagtgagaagctaagcaccagcctctgctgggctgcagaagcggcggcggcggcagcagcagcagcagcatcaggaaggcgctcgggccagcgcggtgaacccgggctgggcagcaggtcgcggagccgcgagccaggatggaggcagagggcagcagcgcgccggcccgggcgggcagcggagagggcagcgacagcgccggcggggccacgctcaaagcccccaagcatctctggaggcacgagcagcaccaccagtacccgctccggcagccccagttccgcctcctgcatccccatcaccacctgcccccgccgccgccaccctcgccccagccccagccccagtgtccgctacagccgccgccgccgccccccctgccgccgcccccgccgccgcccggggctgcccgcggccgctacgcctcgagcggggccaccggccgcgtccggcatcgcggctactcggacaccgagcgctacctgtactgtcgcgccatggaccgcacctcctacgcggtggagaccggccaccggcccggcctgaagaaatccaggatgtcctggccctcctcgttccagggactcaggcgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 11 - protein, 809 aa, Homo sapiens
MEAEGSSAPARAGSGEGSDSAGGATLKAPKHLWRHEQHHQYPLRQPQFRLLHPHHHLPPPPPPSPQPQPQCPLQPPPPPPLPPPPPPPGAARGRYASSGATGRVRHRGYSDTERYLYCRAMDRTSYAVETGHRPGLKKSRMSWPSSFQGLRRFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 12 - DNA, 7979 bp, Homo sapiens
cagcagcaggctcagacctgcttccctggacatttccgggaccgtgagcgagggaaccacgttgccctggattcttgccagctgtacaaagttgaccaggaaaatggctcagcagacaagcccggacactttaacagtacctgaagtggataatccgcattgtccaaacccgtggctgaacgaagaccttgtgaaatccttgcgagaaaacctgttgcagcatgagaagtccaagacagcgaggaaatcggtttctcccaagctctctccagtgatctctccgagaaattcccccaggcttctgcgcagaatgcttctcagcagcaacatccccaaacagcggcgtttcacggtggcacatacatgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 13 - protein, 745 aa, Homo sapiens
MAQQTSPDTLTVPEVDNPHCPNPWLNEDLVKSLRENLLQHEKSKTARKSVSPKLSPVISPRNSPRLLRRMLLSSNIPKQRRFTVAHTCFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 14 - DNA, 19 bp, Artificial Sequence
  PDE4D5_forward primer
gcttctcagcagcaacatc

SEQ ID NO: 15 - DNA, 20 bp, Artificial Sequence
  PDE4D5_reverse primer
tgccattgtccacatcaaaa

SEQ ID NO: 16 - DNA, 24 bp, Artificial Sequence
  PDE4D5 probe
acagcggcgtttcacggtggcaca

SEQ ID NO: 17 - DNA, 7591 bp, Homo sapiens
agttccttatttggtagcttttgacaggactagcctttcttgcaactaagcatcttgacatacattattcattaagccctggagctcgggagagaaagatgcagacccttagatctttagatattcctttatcacgtggattttctttattcagaatagttgctgaattttgtgccattctggagtcttacaaatggcatgtattcgatgggaagacggctggatgggatttaatgcgaggctttcttatgtatacttaattaccaaaaatctttaaaaactcatactctgcgtggcttgtggaggttgttaaagtgtcgagattttgaagctaaatacattttagagcttactatatatatacatatatatatatatacatataatcaatcaaaaatgcctgaagcaaactatttactgtcagtgtcttggggctacataaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 18 - protein, 518 aa, Homo sapiens
MPEANYLLSVSWGYIKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 19 - DNA, 8130 bp, Homo sapiens
agattatagcccagcgtacgagaagcacgagtcctatagttggcgtaccctgaggcctgccagttcctgccttaatgcatatgtagtcgtaattgagttctgacacggccttggatgtttctgtcctaaatagctgacattgcatcttcaagactgtcattccagttggcttttgagtggatacgtgcagtgagatcattgacactggaaacactagttcccattttaattacttaaaacaccacgatgaaaagaaatacctgtgatttgctttctcggagcaaaagtgcctctgaggaaacactacattccagtaatgaagaggaagaccctttccgcggaatggaaccctatcttgtccggagactttcatgtcgcaatattcagcttccccctctcgccttcagacagttggaacaagctgacttgaaaagtgaatcagagaacattcaacgaccaaccagcctccccctgaagattctgccgctgattgctatcacttctgcagaatccagtggttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaa

SEQ ID NO: 20 - protein, 748 aa, Homo sapiens
MKRNTCDLLSRSKSASEETLHSSNEEEDPFRGMEPYLVRRLSCRNIQLPPLAFRQLEQADLKSESENIQRPTSLPLKILPLIAITSAESSGFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 21 - DNA, 20 bp, Artificial Sequence
  PDE4D7_forward primer
gaacattcaacgaccaacca

SEQ ID NO: 22 - DNA, 20 bp, Artificial Sequence
  PDE4D7_reverse primer
tgccattgtccacatcaaaa

SEQ ID NO: 23 - DNA, 27 bp, Artificial Sequence
  PDE4D7 probe
ctgccgctgattgctatcacttctgca

SEQ ID NO: 24 - DNA, 22 bp, Artificial Sequence
  PDE4D7 Forward Primer 2
cgctgattgctatcacttctgc

SEQ ID NO: 25 - DNA, 24 bp, Artificial Sequence
  PDE4D7 Reverse primer
gtcgttgactgtggacaaaatttg

SEQ ID NO: 26 - DNA, 34 bp, Artificial Sequence
  PDE4D7 Probe 2
ttcccttggatcccatgaccagcccataagggaa

SEQ ID NO: 27 - DNA, 8130 bp, Homo sapiens; identical to SEQ ID NO: 19

SEQ ID NO: 28 - protein, 687 aa, Homo sapiens
MAFVWDPLGATVPGPSTRAKSRLRFSKSYSFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 29 - DNA, 8395 bp, Homo sapiens
ttctcactgccctgcggtgttttgaactgccttcttacagacgtcatacagcccttgaggaatagtttctgcctggtgagattgaatgatagttctcattcacaaaaccctggattctaagcagggacacacagaaattactttcgcaggtaaatcagcccacccagccaaagtgtggagagatttgttccttggctgacttctttgctccacggagaggagtgttttcctgtgcttgccctgaaatggaacttccttgacagctctcccgtgttacagtacctcccggtcattttctttttctctctctctacctgcgctcttcgagtgtcagaaacctttaaagctgttactatggaattgcaaaaaagagatcaagtgactctttcactatgctggtttcccttgtgacccagatgaagaatcaattcagaattcagttcctcccttggcattgcaagacacagaagaaactgtcacttcctaacagcctagtactggagtaaattcagtatgaaggaagaaagcgctcctgcgtgttagaaccttgcccatgagctggaccgaggacaggagatggactccaggaaaattggatttcttcaagcagcctcccttggaaatggaatatctttaaaatcttctttgcagaaagacagttagaatgtattaatcagaatagttgaagacttattttcctttttattttttttcaaaatgagcattattatgaagccaagatcccgatctacaagttccctaaggactgcagaggcagtttgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggagatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagtaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacagccaactaggcagtttttaagaatattaacaatatattaacaaacaccaatacaactaatcctatttggttttaatgatttcaccatgggattaagaactatatcaggaacatccctgagaaacggttttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatgggatggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactggggaaatatattctttcccagtgattataaacaatctttttcttttttttaagtccttttggcttctagagctcataggaaaatggacttgatttgaaattggagccagagtttactcgtgttggttatctattcatcagcttcctgacatgttaagagaatacattaaagagaaaatactgttttttaatcctaaaatttttcttccactaagataaaccaaatgtccttacatatatgtaaacccatctatttaaacgcaaaggtgggttgatgtcagtttacatagcagaaagcattcactatcctctaagatttgtttctgcaaaactttcattgctttagaattttaaaatttcaccttgtacaatggccagcccctaaagcaggaaacatttataatggattatatggaaacatcctcccagtacttgcccagcccttgaatcatgtggcttttcagtgaaaggaaagattctttttctaggaaaaatgagcctattttattttattttattttattttttgacacaaactgtagattttagcagccctggcccaaaggaatttgattacttttgttttaaacagtacaaaggggacactataattacaaaaacatccttaactgatttgagttgtttttatttctttggatatattttcagagtggtaaattgtgtgtgagaattacaaatgattattcttttagtggtttcttagcctctcttacagcccacggggatagtactgtacatcaataccttcatatgaaatttttatatgcaatgaaaataaaagcatgggttgattctgcctatttatgactcaatcttttacaaataaaagattattcattttaaattatagttcaatcagcatgtctcttaggatactgaacgtggttgaaatgaaaggatagtgacatcataagttagtactgatattcataaccaaataaagccaacttgagtaattttgctacattaaaaattaccaaaattacttagatggcctataagattaagcatggtgttttctaagcaagctttgaaaggggccttccatacttacttaattgaatattctgggatattgaaaattattcagatacttgacaattatttttggttacctactccgcaaactacaaagttttaaggactcaacaataagttaatgagacacagtgtttgctttcatggagcttacagtctggaggggacaaaggcttaaacaatactcatataattatatatgtgatcagtacaatgaaggagctcagtggggtaaataagcaggaacctgaacttgatctgttccggagggccacagaaggcttccttgaggccttgagaaagtgatttgcatctgagttctgaaggattgtaagaggtaactagggaaaaagttgacaggaagaggaaggggatccagacaagaaacatttgcaaagatcttgaggcataaatgagcttgagacatctggagaaactgaggaaaagtgagagagtaggcagggcctggagccgcagagccattgctaaccatcctgtgtgagatatcccccattctgtagctttattctcataaccctgctcaattttctttataacacttctcacagatttatatacgtgtttgtttttgttatctgtctctcccaccagaccacagctccatgagagcaaggtctttgcttaccaatatatcactagcacttaaaactatgcctggtacacagtaggttcttaatatgtgttgaatatagccatcaaattgatattggatataattcaatctgataagatattttgagatattaaagagtttttaacttgataccataaaaaaaaaaaaaaaaa

SEQ ID NO: 30 - protein, 679 aa, Homo sapiens
MSIIMKPRSRSTSSLRTAEAVCFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 31 - DNA, 26 bp, Artificial Sequence
  PDE4D9_forward primer
atgagcattattatgaagccaagatc

SEQ ID NO: 32 - DNA, 22 bp, Artificial Sequence
  PDE4D9_reverse primer
gtgccattgtccacatcaaaac

SEQ ID NO: 33 - DNA, 27 bp, Artificial Sequence
  PDE4D9 probe
ctacaagttccctaaggactgcagagg

SEQ ID NO: 34 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 35 - protein, 218 aa, Homo sapiens
MATRSPGVVISDDEPGYDLDLFCIPNHYAEDLERVFIPHGLIMDRTERLARDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVRQYNPKMVKVASLLVKRTPRSVGYKPDFVGFEIPDKFVVGYALDYNEYFRDLNHVCVISETGKAKYKA

SEQ ID NO: 36 - DNA, 24 bp, Artificial Sequence
  HPRT1_forward primer
gaggatttggaaagggtgtttatt

SEQ ID NO: 37 - DNA, 21 bp, Artificial Sequence
  HPRT1_reverse primer
acagagggctacaatgtgatg

SEQ ID NO: 38 - DNA, 26 bp, Artificial Sequence
  HPRT1 probe
acgtcttgctcgagatgtgatgaagg

SEQ ID NO: 39 - DNA, 1771 bp, Homo sapiens
ggcggccaggccgggcgcggagtgggcgcgcggggccggaggaggggccagcgaccgcggcaccgcctgtgcccgcccgcccctccgcagccgctacttaagaggctccagcgccggccccgccctagtgcgttacttacctcgactcttagcttgtcggggacggtaaccgggacccggtgtctgctcctgtcgccttcgcctcctaatccctagccactatgcgtgagtgcatctccatccacgttggccaggctggtgtccagattggcaatgcctgctgggagctctactgcctggaacacggcatccagcccgatggccagatgccaagtgacaagaccattgggggaggagatgactccttcaacaccttcttcagtgagacgggcgctggcaagcacgtgccccgggctgtgtttgtagacttggaacccacagtcattgatgaagttcgcactggcacctaccgccagctcttccaccctgagcagctcatcacaggcaaggaagatgctgccaataactatgcccgagggcactacaccattggcaaggagatcattgaccttgtgttggaccgaattcgcaagctggctgaccagtgcaccggtcttcagggcttcttggttttccacagctttggtgggggaactggttctgggttcacctccctgctcatggaacgtctctcagttgattatggcaagaagtccaagctggagttctccatttacccagcaccccaggtttccacagctgtagttgagccctacaactccatcctcaccacccacaccaccctggagcactctgattgtgccttcatggtagacaatgaggccatctatgacatctgtcgtagaaacctcgatatcgagcgcccaacctacactaaccttaaccgccttattagccagattgtgtcctccatcactgcttccctgagatttgatggagccctgaatgttgacctgacagaattccagaccaacctggtgccctacccccgcatccacttccctctggccacatatgcccctgtcatctctgctgagaaagcctaccatgaacagctttctgtagcagagatcaccaatgcttgctttgagccagccaaccagatggtgaaatgtgaccctcgccatggtaaatacatggcttgctgcctgttgtaccgtggtgacgtggttcccaaagatgtcaatgctgccattgccaccatcaaaaccaagcgcagcatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaattatccattccttttggccctgcagcatgtcatgctcccagaatttcagcttcagcttaactgacagacgttaaagctttctggttagattgttttcacttggtgatcatgtcttttccatgtgtacctgtaatatttttccatcatatctcaaagtaaagtcattaacatcaaaaaaaaaaaaaaaaaaaaaa

SEQ ID NO: 40 - protein, 451 aa, Homo sapiens [sequence omitted]

SEQ ID NO: 41 - DNA, 22 bp, Artificial Sequence
  TUBA1B_forward primer
tgactccttcaacaccttcttc

SEQ ID NO: 42 - DNA, 18 bp, Artificial Sequence
  TUBA1B_reverse primer
tgccagtgcgaacttcat

SEQ ID NO: 43 - DNA, 24 bp, Artificial Sequence
  TUBA1B probe
ccgggctgtgtttgtagacttgga

SEQ ID NO: 44 - DNA, 5416 bp, Homo sapiens
agtgggccgccatgttgtcggagtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcaggtaattaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 45 - DNA, 5410 bp, Homo sapiens
agtgggccgccatgttgtcggagtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcagaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 46 - protein, 1188 aa, Homo sapiens
MSVACVLKRKAVLWQDSFSPHLKHHPQEPANPNMPVVLTSGTGSQAQPQPAANQALAAGTHSSPVPGSIGVAGRSQDDAMVDYFFQRQHGEQLGGGGSGGGGYNNSKHRWPTGDNIHAEHQVRSMDELNHDFQALALEGRAMGEQLLPGKKFWETDESSKDGPKGIFLGDQWRDSAWGTSDHSVSQPIMVQRRPGQSFHVNSEVNSVLSPRSESGGLGVSMVEYVLSSSPGDSCLRKGGFGPRDADSDENDKGEKKNKGTFDGDKLGDLKEEGDVMDKTNGLPVQNGIDADVKDFSRTPGNCQNSANEVDLLGPNQNGSEGLAQLTSTNGAKPVEDFSNMESQSVPLDPMEHVGMEPLQFDYSGTQVPVDSAAATVGLFDYNSQQQLFQRPNALAVQQLTAAQQQQYALAAAHQPHIGLAPAAFVPNPYIISAAPPGTDPYTAGLAAAATLGPAVVPHQYYGVTPWGVYPASLFQQQAAAAAAATNSANQQTTPQAQQGQQQVLRGGASQRPLTPNQNQQGQQTDPLVAAAAVNSALAFGQGLAAGMPGYPVLAPAAYYDQTGALVVNAGARNGLGAPVRLVAPAPVIISSSAAQAAVAAAAASANGAAGGLAGTTNGPFRPLGTQQPQPQPQQQPNNNLASSSFYGNNSLNSNSQSSSLFSQGSAQPANTSLGFGSSSSLGATLGSALGGFGTAVANSNTGSGSRRDSLTGSSDLYKRTSSSLTPIGHSFYNGLSFSSSPGPVGMPLPSQGPGHSQTPPPSLSSHGSSSSLNLGGLTNGSGRYISAAPGAEAKYRSASSASSLFSPSSTLFSSSRLRYGMSDVMPSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQVINEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGPICGPPNGII

SEQ ID NO: 47 - protein, 1186 aa, Homo sapiens
MSVACVLKRKAVLWQDSFSPHLKHHPQEPANPNMPVVLTSGTGSQAQPQPAANQALAAGTHSSPVPGSIGVAGRSQDDAMVDYFFQRQHGEQLGGGGSGGGGYNNSKHRWPTGDNIHAEHQVRSMDELNHDFQALALEGRAMGEQLLPGKKFWETDESSKDGPKGIFLGDQWRDSAWGTSDHSVSQPIMVQRRPGQSFHVNSEVNSVLSPRSESGGLGVSMVEYVLSSSPGDSCLRKGGFGPRDADSDENDKGEKKNKGTFDGDKLGDLKEEGDVMDKTNGLPVQNGIDADVKDFSRTPGNCQNSANEVDLLGPNQNGSEGLAQLTSTNGAKPVEDFSNMESQSVPLDPMEHVGMEPLQFDYSGTQVPVDSAAATVGLFDYNSQQQLFQRPNALAVQQLTAAQQQQYALAAAHQPHIGLAPAAFVPNPYIISAAPPGTDPYTAGLAAAATLGPAVVPHQYYGVTPWGVYPASLFQQQAAAAAAATNSANQQTTPQAQQGQQQVLRGGASQRPLTPNQNQQGQQTDPLVAAAAVNSALAFGQGLAAGMPGYPVLAPAAYYDQTGALVVNAGARNGLGAPVRLVAPAPVIISSSAAQAAVAAAAASANGAAGGLAGTTNGPFRPLGTQQPQPQPQQQPNNNLASSSFYGNNSLNSNSQSSSLFSQGSAQPANTSLGFGSSSSLGATLGSALGGFGTAVANSNTGSGSRRDSLTGSSDLYKRTSSSLTPIGHSFYNGLSFSSSPGPVGMPLPSQGPGHSQTPPPSLSSHGSSSSLNLGGLTNGSGRYISAAPGAEAKYRSASSASSLFSPSSTLFSSSRLRYGMSDVMPSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGPICGPPNGII

SEQ ID NO: 48 - DNA, 22 bp, Artificial Sequence
  PUM1_forward primer
gccagcttgtcttcaatgaaat

SEQ ID NO: 49 - DNA, 21 bp, Artificial Sequence
  PUM1_reverse primer
caaagccagcttctgttcaag

SEQ ID NO: 50 - DNA, 24 bp, Artificial Sequence
  PUM1 probe
atccaccatgagttggtaggcagc

SEQ ID NO: 51 - DNA, 1921 bp, Homo sapiens
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 52 - protein, 339 aa, Homo sapiens
MDQNNSLPPYAQGLASPQGAMTPGIPIFSPMMPYGTGLTPQPIQNTNSLSILEEQQRQQQQQQQQQQQQQQQQQQQQQQQQQQQQQQQQQQQQQQAVAAAAVQQSTSQQATQGTSGQAPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 53 - DNA, 23 bp, Artificial Sequence
  TBP_forward primer
gccaagaagaaagtgaacatcat

SEQ ID NO: 54 - DNA, 20 bp, Artificial Sequence
  TBP_reverse primer
atagggattccgggagtcat

SEQ ID NO: 55 - DNA, 24 bp, Artificial Sequence
  TBP probe
tcagaacaacagcctgccacctta

SEQ ID NO: 56 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 57 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 58 - DNA, 18 bp, Artificial Sequence
  ACTB_forward primer
ccaaccgcgagaagatga

SEQ ID NO: 59 - DNA, 20 bp, Artificial Sequence
  ACTB_reverse primer
ccagaggcgtacagggatag

SEQ ID NO: 60 - DNA, 23 bp, Artificial Sequence
  ACTB probe
ccatgtacgttgctatccaggct

SEQ ID NO: 61 - DNA, 1229 bp, Homo sapiens
gtctgacgggcgatggcgcagccaatagacaggagcgctatccgcggtttctgattggctactttgttcgcattataaaaggcacgcgcgggcgcgaggcccttctctcgccaggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 62 - protein, 317 aa, Homo sapiens
MPREDRATWKSNYFLKIIQLLDDYPKCFIVGADNVGSKQMQQIRMSLRGKAVVLMGKNTMMRKAIRGHLENNPALEKLLPHIRGNVGFVFTKEDLTEIRDMLLANKVPAAARAGAIAPCEVTVPAQNTGLGPEKTSFFQALGITTKISRGTIEILSDVQLIKTGDKVGASEATLLNMLNISPFSFGLVIQQVFDNGSIYNPEVLDITEETLHSRFLEGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

SEQ ID NO: 63 - protein, 317 aa, Homo sapiens; identical to SEQ ID NO: 62

SEQ ID NO: 64 - DNA, 18 bp, Artificial Sequence
  RPLP0_forward primer
taaaccctgcgtggcaat

SEQ ID NO: 65 - DNA, 27 bp, Artificial Sequence
  RPLP0_reverse primer
acatttcggataatcatccaatagttg

SEQ ID NO: 66 - DNA, 24 bp, Artificial Sequence
  RPLP0 probe
aagtagttggacttccaggtcgcc

SEQ ID NO: 67 - DNA, 2458 bp, Homo sapiens
cagaagaaggcagcgcccaaggcgcatgcgcagcggtcactcccgctgtatattaaggcgccggcgatcgcggcctgaggctgctcccggacaagggcaacgagcgtttcgtttggacttctcgacttgagtgcccgcctccttcgccgccgcctctgcagtcctcagcgcagttatgcccagttcttcccgctgtggggacacgaccacggaggaatccttgcttcagggactcgggaccctgctggaccccttcctcgggtttaggggatgtggggaccaggagaaagtcaggatccctaagagtcttccctgcctggatggatgagtggcttcttctccacctagattctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacatcatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtgaaaaaa

SEQ ID NO: 68 - DNA, 2281 bp, Homo sapiens
cagaagaaggcagcgcccaaggcgcatgcgcagcggtcactcccgctgtatattaaggcgccggcgatcgcggcctgaggctgctcccggacaagggcaacgagcgtttcgtttggacttctcgacttgagtgcccgcctccttcgccgccgcctctgcagtcctcagcgcagtctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacatcatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtgaaaaaa

SEQ ID NO: 69 - protein, 640 aa, Homo sapiens
MESVVRRCPFLSRVPQAFLQKAGKSLLFYAQNCPKMMEVGAKPAPRALSTAAVHYQQIKETPPASEKDKTAKAKVQQTPDGSQQSPDGTQLPSGHPLPATSQGTASKCPFLAAQMNQRGSSVFCKASLELQEDVQEMNAVRKEVAETSAGPSVVSVKTDGGDPSGLLKNFQDIMQKQRPERVSHLLQDNLPKSVSTFQYDRFFEKKIDEKKNDHTYRVFKTVNRRAHIFPMADDYSDSLITKKQVSVWCSNDYLGMSRHPRVCGAVMDTLKQHGAGAGGTRNISGTSKFHVDLERELADLHGKDAALLFSSCFVANDSTLFTLAKMMPGCEIYSDSGNHASMIQGIRNSRVPKYIFRHNDVSHLRELLQRSDPSVPKIVAFETVHSMDGAVCPLEELCDVAHEFGAITFVDEVHAVGLYGARGGGIGDRDGVMPKMDIISGTLGKAFGCVGGYIASTSSLIDTVRSYAAGFIFTTSLPPMLLAGALESVRILKSAEGRVLRRQHQRNVKLMRQMLMDAGLPVVHCPSHIIPVRVADAAKNTEVCDELMSRHNIYVQAINYPTVPRGEELLRIAPTPHHTPQMMNYFLENLLVTWKQVGLELKPHSSAECNFCRRPLHFEVMSEREKSYFSGLSKLVSAQA

SEQ ID NO: 70 - protein, 640 aa, Homo sapiens; identical to SEQ ID NO: 69

SEQ ID NO: 71 - DNA, 17 bp, Artificial Sequence
  ALAS-1_forward primer
agccacatcatccctgt

SEQ ID NO: 72 - DNA, 22 bp, Artificial Sequence
  ALAS-1_reverse primer
cgtagatgttatgtctgctcat

SEQ ID NO: 73 - DNA, 22 bp, Artificial Sequence
  ALAS-1 probe
tttagcagcatctgcaacccgc

(19) United States
(12) Patent Application Publication
(10) Pub. No.: US 2021/0277479 A1
(43) Pub. Date: Sep. 9, 2021

(54) RISK SCORES BASED ON HUMAN PHOSPHODIESTERASE 4 D VARIANT 7 EXPRESSION

(71) Applicant: KONINKLIJKE PHILIPS N.V., EINDHOVEN (NL)

(72) Inventors: Ralf HOFFMAN, BRUEGGEN (DE); Dianne Arnoldina Margaretha Wilhelmina VAN STRIJP, `s-Hertogenbosch (NL); Anne Godefrida Catharina VAN BRUSSEL, VUGHT (NL); Janneke WROBEL, EINDHOVEN (NL); Johannes Baptist Adrianus Dionisius VAN ZON, WAALRE (NL); Eveline Catharina Anna Clasina DEN BIEZEN, DONGEN (NL); Márcia ALVES DE INDA, ROSMALEN (NL)

(21) Appl. No.: 16/461,025

(22) PCT Filed: Nov. 28, 2017
(86) PCT No.: PCT/EP2017/080591

§ 371 Date: May 15, 2019

(30) Foreign Application Priority Data
Dec. 1, 2016 (EP) 16201712.3

(51) Int. Cl. C12Q 1/6886; G16B 20/20

(52) U.S. Cl. CPC C12Q 1/6886; G16B 20/20; C12Q 2600/106; C12Q 2600/158; C12Q 2600/118

(57) ABSTRACT

Methods are described for stratifying patient risk for patients with prostate cancer and for providing a treatment recommendation to a patient based on a phosphodiesterase 4D variant 7 (PDE4D7) risk score. A diagnostic kit and a computer program product for the analysis and determination of the PDE4D7 risk score are also described.

### BACKGROUND

Cancer is a class of diseases in which a group of cells display uncontrolled growth, invasion and sometimes metastasis. These three malignant properties of cancers differentiate them from benign tumors, which are self-limited and do not invade or metastasize. Prostate Cancer (PCa) is the most commonly-occurring non-skin malignancy in men. Due to ageing populations, the incidence of PCa is expected to dramatically increase in the future. Routine diagnosis by determination of blood levels of the prostate-specific antigen (PSA), digital rectal exam (DRE) and transrectal ultrasound analysis (TRUS) leads to a significant over-diagnosis of non-cancerous, benign prostate conditions. Of the approximately 1 million prostate biopsies annually performed in the U.S. to find about 250,000 new cases, about 75% are done unnecessarily, incurring both substantial complications (such as urosepsis, bleeding, and urinary retention) in patients and a high cost. At least 4 out of 100 men with a negative biopsy are likely to be hospitalized due to side-effects and 9 out of 10,000 biopsied patients are at risk of dying from the currently used procedure.

Of the approximately 250,000 newly detected PCa cases in the U.S. per year, about 200,000 are initially characterized as localized disease, i.e., as cancer confined to the prostate organ. This condition is, to a certain extent, curable by primary treatment approaches, such as radiation therapy or the partial or total removal of the prostate by surgery (prostatectomy). However, these interventions typically come with serious side effects, particularly urinary incontinence and/or erectile dysfunctions as very frequent consequences of prostatectomy. Further, the routinely-applied treatments for localized PCa are expensive.

Among the approximately 200,000 men in the United States with clinically localized disease at diagnosis, up to 50% have very-low- or low-risk cancer. Accordingly, the National Comprehensive Cancer Network (NCCN) recently revised their PCa treatment guidelines to expand active surveillance (AS) as a gentle and convenient treatment alternative for patients with such low risk disease. By referring appropriate patients to AS, the quality of life for such patients is significantly improved as compared with men having undergone primary treatment and the 5-year cost for AS is reported to be significantly lower, per patient.

Moreover, in case surgery (vs. AS) is selected as the treatment of choice for a given patient, it is of significant advantage to stratify for the extent of surgery according to the potential aggressiveness of the patient's tumor. For instance, nerve-sparing operation techniques could be more generally applied for men with predicted low-risk disease to minimize potency-related adverse effects of radical prostatectomy. Likewise, according to the European Association Of Urology (EAU)'s latest Prostate Cancer Guidelines, extended lymph node dissection is recommended in case of a predicted high-risk cancer despite the fact that the procedure is complex, time-consuming and associated with higher complication rates as compared with more limited procedures. Consequently, while less limited lymph node dissection has shown to miss about 50% of lymph node metastases, the treatment management for men with localized prostate cancer would benefit from highly accurate pre-surgical predictions of the aggressiveness potential of an individual tumor to provide the optimal care for each patient.

The side effects of active treatment options (e.g., surgery, radiation therapy, etc.) can be avoided or reduced by the selection of active surveillance as a treatment alternative. However, as the tumor is not treated while in active surveillance, the likelihood of disease progression should be very minimal to ensure that the number of patients who may progress under active surveillance still have a good chance of being cured by switching from active surveillance to active intervention. Traditional methods of determining patient risk of disease progression tend to assign many patients to the active intervention categories rather than AS, thereby reducing the patient's quality of life and unnecessarily subjecting such patients to the adverse side-effects of invasive treatments. Thus, new methods of stratifying patient risk and providing improved recommendations to patients on whether to select active surveillance versus active intervention are desirable.

WO 2010/131194 A1 discloses a method for diagnosing or detecting malignant, hormone sensitive prostate cancer by determining the expression level of the phosphodiesterase 4D variant PDE4D7. The document also discloses the use of a PDE-Index to discriminate effectively between benign and malignant diseases, in which the expression of PDE4D7 is normalized against PDE4D5 as an internal control.

WO 2010/131195 A1 describes a method for diagnosing hormone resistant vs. hormone sensitive prostate cancer by determining the expression level of PDE4D7. The PDE4D7 expression level is normalized to a reference gene, which may be PDE4D5.

In Henderson, et al., “The cAMP phosphodiesterase-4D7 (PDE4D7) is downregulated in androgen-independent prostate cancer cells and mediates proliferation by compartmentalizing cAMP at the plasma membrane of VCaP prostate cancer cells” British Journal of Cancer, 110(5) 1278-1287 (2014), evidence is presented for PDE4D7 being highly expressed in androgen sensitive prostate cancer cells while being significantly downregulated in androgen insensitive prostate cancer cells and suggests a potential application as a biomarker for androgen insensitive prostate cancer as well as therapeutic possibilities.

EP 1471153 A2 describes a transcriptional activity assay for determining the biological activity of a compound by analyzing its capability to modulate gene expression. Among the possible target expression products are PDE4D isoenzymes. The compounds identified in the described screenings may be antibodies, which are of therapeutic value in the treatment of breast cancer.

WO 2010/059838 A2 describes inhibitors of phosphodiesterase-4 (PDE4) and their use in the treatment and prevention of stroke, myocardial infarction, cardiovascular inflammatory diseases and disorders as well as central nervous system disorders.

WO 2004/090157 A1 discloses the use of PDE4D, in particular PDE4D5 or PDE4D7, as a target for the identification of compounds that can be used for the treatment of atherosclerosis or for the treatment of restenosis.

US 2003/220273 A1 describes antisense compounds, compositions and methods for modulating the expression of phosphodiesterase 4D and the use of these compounds for treatment of diseases associated with expression of phosphodiesterase 4D.

Merkle, et al., “Roles of cAMP and cAMP-dependent protein kinase in the progression of prostate cancer: Cross-talk with the androgen receptor” Cellular Signalling, 23(3) 507-515, (2011) describes a study on the roles of cAMP and cAMP-dependent protein kinase in the progression of prostate cancer. In the context of this study it is stated, that PDE4D expression is increased in cancer tissues.

### BRIEF DESCRIPTION

The present invention relates to methods for diagnosing, monitoring, or prognosticating prostate cancer or the progression state of prostate cancer. In particular, it relates to a method for risk stratification for therapy selection in a patient with prostate cancer based on the expression level of a PDE4D variant, such as PDE4D7, and to a diagnostic kit used to determine a risk score for men with prostate cancer. PDE4D7 refers to a cyclic nucleotide phosphodiesterase (PDE), of the cyclic adenosine monophosphate (cAMP) family (4), isoform D, variant 7.

In accordance with one aspect of the exemplary embodiment, the method of risk stratification includes determining a normalized gene expression profile for a single marker gene consisting of phosphodiesterase 4D variant 7 (PDE4D7), with respect to a set of reference genes selected from the group consisting of Homo sapiens hypoxanthine phosphoribosyltransferase 1 (HPRT1), Tubulin-Alpha-1b (TUBA1B) Homo sapiens pumilio RNA-Binding Family Member (PUM1) and Homo sapiens TATA box binding protein (TBP), and combinations thereof. The method further includes determining a prognostic risk score with a scoring function, based on the normalized gene expression profile, the scoring function having been derived from gene expression profiles for biological samples taken from subjects that have been monitored for prostate cancer. The method may further include determining a combined prognostic risk score based on the prognostic risk score and a second risk determination.

In accordance with another aspect, a diagnostic kit includes at least one primer and/or probe for determining the expression level of at least one phosphodiesterase 4D (PDE4D) variant, the at least one PDE4D variant comprising PDE4D7, and at least one primer and/or probe for determining the gene expression level of at least one reference gene. The kit optionally includes instructions for computing a risk score based on the determined expression levels. Optionally, the instructions are stored on a computer program product which, when executed by a computer, perform a method that includes determining a normalized gene expression profile for the phosphodiesterase 4D variant 7 (PDE4D7), with respect to the at least one reference gene and determining a prognostic risk score with a scoring function, based on the normalized gene expression profile. Optionally, instructions are stored on a computer program product which, when executed by a computer, compute a combined prognostic risk score based on the prognostic risk score and a second risk determination.

In accordance with another aspect, a method of providing a therapy recommendation for a subject with prostate cancer includes determining a gene expression profile of a biological sample from the subject. The gene expression profile includes an expression level for phosphodiesterase 4D variant 7 (PDE4D7). The gene expression profile is normalized using an expression level for at least one reference gene selected from HPRT1, TUBA1B, PUM1, and TBP. A prognostic risk score is determined for the subject based on the normalized gene expression profile. The subject is categorized into a PDE4D7 risk group, based on the prognostic risk score. A therapy recommendation is provided for the subject, based on the PDE4D7 risk group. The method may also comprise determining a combined prognostic risk score for the subject based on the prognostic risk score and a second risk determination for the subject. The subject is categorized into a risk group, based on the combined prognostic risk score. A therapy recommendation is provided for the subject, based on the risk group.

In accordance with another aspect, a computer program product includes a non-transitory recording medium storing instructions, which when executed on a computer, cause the computer to perform a method including computing a normalized gene expression profile for phosphodiesterase 4D variant 7 (PDE4D7), with respect to a set of reference genes selected from the group consisting of: Homo sapiens hypoxanthine phosphoribosyltransferase 1 (HPRT1), Tubulin-Alpha-1b (TUBA1B) Homo sapiens pumilio RNA-Binding Family Member (PUM1) and Homo sapiens TATA box binding protein (TBP), and combinations thereof; and computing a prognostic risk score for the subject based on the gene expression profile with a scoring function. The scoring function may have been derived from gene expression profiles for biological samples taken from subjects that have been monitored for prostate cancer. The method may further include computing a combined prognostic risk score based on the prognostic risk score and a second risk determination.

The second risk determination is a risk determination other than the PDE4D7 risk score (prognostic risk score), e.g. it may be based on a Gleason score. Preferably, the second risk determination is a National Comprehensive Cancer Network (NCCN) classification, such as one or more of very low risk (VLR), low risk (LR), favorable intermediate risk (FIR), unfavorable intermediate risk (UIR), and high risk (HR). The combined prognostic risk score may be determined with a regression function derived from subjects that have been monitored for prostate cancer.

In some embodiments of any of the above aspects, the gene expression profile is converted into at least one prostate cancer PDE risk score (prognostic risk score) indicative for the presence and/or absence of prostate cancer and/or the prostate cancer progression state. The introduction of the PDE risk score provides a good predication in prostate cancer diagnosis or prognosis. Specifically, the PDE4D7 risk score can be used to stratify subjects with prostate cancer based on the measured level of this risk score, indicating whether to place such subjects on active surveillance (AS) rather than active treatment (e.g., surgery, radiation therapy, etc.), which is the standard of care for these subjects.

The gene expression profile may further include an expression level for one or more other PDE4D variants. For example, the other PDE4D variant(s) may include one or more of PDE4D1, PDE4D2, PDE4D3, PDE4D4, PDE4D5, PDE4D6, PDE4D8 and PDE4D9.

The gene expression profile may be a gene expression profile of a biological sample from an individual, such as a biopsy from an individual's prostate.

The gene expression profile may be a normalized gene expression profile that is obtained by normalizing the expression level of at least the PDE4D7 variant to the expression of at least one reference gene. The method may include determining the expression level of one or more reference genes in a sample before normalizing the expression level of at least the PDE4D7 variant.

The reference gene(s) may be selected from Homo sapiens hypoxanthine phosphoribosyltransferase 1 (HPRT1), Tubulin-Alpha-1b (TUBA1B), Homo sapiens pumilio RNA-Binding Family Member (PUM1), and Homo sapiens TATA box binding protein (TBP), and combinations thereof, such as at least two, or at least three, or all of these.

The prognostic risk score may be based on the normalized gene expression profile that includes the expression level for PDE4D7.

The gene expression level may be determined by detecting mRNA expression using one or more primers and/or probes and/or one or more sets thereof.

The gene expression level may be determined by an amplification based method and/or microarray analysis and/or RNA sequencing.

The determining of the gene expression profile may include performing Real-Time Quantitative Polymerase Chain Reaction (RT-qPCR) on RNA extracted from the biological sample. In other embodiments, the gene expression level is determined by RNA sequencing, conventional PCR (using, e.g., end point analysis by gel electrophoresis), or multiplex-PCR.

In the case of RT-qPCR, the determining of the gene expression profile may include determining a threshold cycle (Cₜ) value for PDE4D7 and each of the at least one reference genes.

The determining of the prognostic risk score may include normalizing the PDE4D7 value, using the value of each of the at least one reference genes. The determining of the prognostic risk score may include computing the risk score as a function, such as a linear function, of the normalized value. The function may be derived based on outcomes of patients following acquisition of a biological sample.

The PCR may be performed with at least one primer and/or probe for measuring a reference gene selected from HPRT1, TUBA1B, PUM1, and TBP.

The prognostic risk score for the subject may be a value in a pre-defined range.

The method may further include categorizing the subject into one of a predefined set of risk groups, based on the prognostic risk score or the combined prognostic risk score. There may be at least two or at least three risk groups based on the prognostic risk score or the combined prognostic risk score.

The method may further include at least one of: a) proposing a therapy for the subject based on the assigned risk group, wherein at least two of the risk groups are associated with different potential therapies; b) computing a disease progression risk prediction of the subject before or after prostate surgery; and c) computing a therapy response prediction for the subject before or after prostate surgery. In the case of proposing a therapy, the proposed therapies may be selected from: a) at least a partial prostatectomy; b) an active therapy selected from radiation treatment, hormone therapy, chemotherapy, and a combination thereof; and c) observation without performing a) or b). The proposed therapies may include: prostate surgery, prostate removal, chemotherapy, radiotherapy, hormone therapy and limited or extended lymph node dissection, or a combination thereof.

The proposed therapy based on the assigned risk group may be different from a proposed therapy based only on the second risk determination.

The method and kit may include a nucleic acid array including one or more oligonucleotide probes complementary and hybridizable to a coding sequence of at least one PDE4D variant selected from PDE4D1, PDE4D2, PDE4D3, PDE4D4, PDE4D5, PDE4D6, PDE4D7, PDE4D8 and PDE4D9, and which may further include one or more oligonucleotide probes complementary and hybridizable to at least one of the reference genes selected from TBP, HPRT1, PUM1, and TUBA1B, for determining a risk score as defined herein.

Another aspect of the exemplary embodiment relates to a use of the PDE4D7 variant and reference genes for risk stratification.

An additional aspect of the invention refers to a computer implemented method for diagnosing, monitoring or prognosticating prostate cancer or stratifying the progression risk of prostate cancer, comprising the method steps as defined herein.

A further aspect of the invention relates to a computer program product including a non-transitory recording medium with instructions stored thereon, which when executed on a computer, cause the computer to perform a method which includes computing a normalized gene expression profile for phosphodiesterase 4D variant 7 (PDE4D7), with respect to a set of reference genes selected from the group consisting of: Homo sapiens hypoxanthine phosphoribosyltransferase 1 (HPRT1), Tubulin-Alpha-1b (TUBA1B) Homo sapiens pumilio RNA-Binding Family Member (PUM1) and Homo sapiens TATA box binding protein (TBP), and combinations thereof, and computing a prognostic risk score for the subject based on the gene expression profile with a scoring function that is derived from gene expression profiles for biological samples taken from subjects that have been monitored for prostate cancer. The method may further include computing a combined prognostic risk score based on the prognostic risk score and a second risk determination.

### BRIEF DESCRIPTION OF THE DRAWINGS

FIG. 1 is a flow chart illustrating a method of risk stratification for therapy selection in a patient with prostate cancer;

FIG. 2 shows the normalized gene expression profile of PDE4D7 (left) and the PDE4D7 risk score transformation (right);

FIG. 3 is a Forest plot of Hazard Ratios (HR) and 95% confidence intervals (95% CI) after multivariate Cox regression analysis of the total patient cohort (including 503 patients), wherein the tested clinical endpoint is the time to biochemical recurrence (BCR) after surgery;

FIG. 4 shows a Kaplan Meier analysis of time to prostate-specific antigen (PSA) relapse after prostatectomy for the PDE4D7 risk score groups. The number of patients (i.e., men) at risk for every 20-month time interval per risk score group and a group-wise comparison of the Hazard Ratios is also shown;

FIG. 5 shows a Forest plot of Hazard Ratios (HR) and 95% confidence intervals (95% CI) after multivariate Cox regression analysis of the total patient cohort (503 patients), wherein the tested clinical endpoint is the time to biochemical recurrence (BCR) after surgery;

FIG. 6 shows a Forest plot of Hazard Ratios and 95% confidence intervals (95% CI) for multiple clinical post-surgical endpoints, including biochemical recurrence, salvage radiation therapy, salvage androgen deprivation therapy, clinical recurrence, prostate cancer specific mortality, and overall mortality;

FIG. 7 shows a graph of the 5-year risk of biochemical recurrence (BCR) in the NCCN risk groups versus the PDE4D7 risk score groups;

FIG. 8 shows a graph of the 10-year risk of clinical recurrence (CR) in the NCCN risk groups versus the PDE4D7 risk score groups;

FIG. 9 shows a graph of the 10-year risk of prostate cancer-specific mortality (PCSM) in the NCCN risk groups versus the PDE4D7 risk score groups.

FIG. 10 shows a graph of the 10-year risk overall mortality (OM) in the NCCN risk groups versus the PDE4D7 risk score groups.

FIG. 11 illustrates a risk progression matrix in the NCCN clinical risk groups versus the PDE4D7 risk groups.

FIG. 12 shows a Kaplan-Meier analysis of the biopsy Gleason score for biochemical recurrence in the NCCN favorable intermediate risk group (128 patients). The biopsy Gleason score s categorized into Gleason grade groups 3+3 (the lower line in the figure) and 3+4 (the upper line in the figure). Also illustrated is a pair-wise risk group comparison of the Hazard Ratios (HR).

FIG. 13 shows a Kaplan-Meier analysis of the PDE4D7 risk score groups for biochemical recurrence in the NCCN favorable intermediate risk group (128 patients). Also illustrated is a pair-wise risk group comparison of the Hazard Ratios (HR).

FIG. 14 shows a Kaplan-Meier analysis of the biopsy Gleason score for biochemical recurrence in the NCCN unfavorable intermediate and high risk group (164 patients). The biopsy Gleason score is categorized into Gleason grade groups 3+3, 3+4, 4+3, and ≥4+4. Also illustrated is a pair-wise risk group comparison of the Hazard Ratios (HR).

FIG. 15 shows a Kaplan-Meier analysis of the PDE4D7 risk score groups for biochemical recurrence in the NCCN unfavorable intermediate & high risk group (164 patients). Also illustrated is a pair-wise risk group comparison of the Hazard Ratios (HR).

FIG. 16 shows a calibration plot of the NCCN & PDE4D7 score logistic regression model to predict 5-year biochemical relapse after surgery based on a contingency table after Hosmer-Lemeshow testing of the 449 patient cohort with complete 5-year follow-up.

FIG. 17 shows a calibration plot of the NCCN & PDE4D7 score logistic regression model to predict 5-year biochemical relapse after surgery based on a contingency table after Hosmer-Lemeshow testing of the 449 patient cohort with complete 5-year follow-up.

FIG. 18 shows a ROC analysis of 2-year biochemical relapse after surgery (BCR) of the NCCN & PDE4D7 score logistic regression model in a patient cohort (449 patients) with complete 5-year follow-up.

FIG. 19 shows a ROC analysis of 5-year biochemical relapse after surgery (BCR) of the NCCN & PDE4D7 score logistic regression model in a patient cohort (449 patients) with complete 5-year follow-up.

FIG. 20 shows a ROC analysis of 10-year biochemical relapse after surgery (BCR) of the NCCN & PDE4D7 score logistic regression model in a patient cohort (379 patients) with complete 10-year follow-up.

FIG. 21 shows a predicted risk analysis per NCCN group as a function of the PDE4D7 risk score that revealed a heterogeneous 5-year progression risk (BCR) distribution even within the lowest NCCN clinical risk groups.

FIG. 22 shows results of a Kaplan-Meier analysis of the biochemical recurrence (BCR) free survival in the patient sub-cohort with 5-year complete follow-up (449 patients). Four risk categories were defined based on the logistic regression model calculated 5-year probability p to experience biochemical recurrence: risk group 1 (0 to <0.25; 139 patients); risk group 2 (0.25 to <0.5; 170 patients); risk group 3 (0.5 to <0.75; 112 patients); risk group 4 (0.75 to 1.0; 28 patients).

### DETAILED DESCRIPTION

Aspects of the exemplary embodiment relate to the identification and use of gene expression profiles, signatures, or patterns of biomarker genes of interest (also referred to as marker genes or GOIs (genes of interest)) with clinical relevance to prostate cancer. In particular, the method uses the gene expression analysis of nucleic acids, such as transcripts of biomarker genes, obtained from biological samples. The expression analysis of these marker genes can be used in providing prostate cancer PDE4D7 risk score for stratifying the patient's risk of reaching certain clinical outcomes.

More specifically, a method is described for the determination of a risk score based on the PDE4D7 expression profile, which has been found to provide a unique means to stratifying a patient's risk of developing particular pre- and post-surgical endpoints, including biochemical recurrence, clinical recurrence, prostate cancer-specific mortality, and overall mortality. The PDE risk score provides a very helpful parameter for personalized medicine relating to the diagnosis, prognosis, and treatment of prostate cancer patients. The PDE risk score may be used alone or in combination with other means and methods that provide information on the patient's personal disease status or disease stage.

Physicians and/or pathologists can advantageously use the PDE risk score to confirm results obtained in other methods for diagnosing, identifying, and prognosticating patients. The methods and means provided by the invention therefore help establish better diagnosis, prognosis, etc. to find the best treatment for a patient, and to avoid unnecessary surgery or other treatments that are dangerous due to side-effects, and result in costs savings.

As used herein, the term “PDE4D transcript variant” or “PDE4D isoform” or “PDE4D variant” relates to any of the PDE4D splice variants of the human phosphodiesterase PDE4D, i.e., the human phosphodiesterase PDE4D gene, for example PDE4D1, PDE4D2, PDE4D3, PDE4D4, PDE4D5, PDE4D6, PDE4D7, PDE4D8 and PDE4D9.

The terms “marker” “maker gene” “GOI” or “PDE4D variant marker,” can be used interchangeably and relate to a gene, genetic unit or sequence (a nucleotide sequence or amino acid or protein sequence) as defined herein above, whose expression level is increased or decreased in malignant or benign, prostate cancer cell or tissue or in any type of sample including such cells or tissues or portions or fragments thereof, when comparing to a control level, when comparing to the expression in normal tissue. The term also refers to any expression product of said genetic unit or sequence, in particular to a PDE4D variant mRNA transcript, a polypeptide or protein encoded by the PDE4D variant transcript or fragments thereof, as well as homologous derivatives thereof as described herein above. In particular, the terms “marker” “marker gene,” “GOI,” or “PDE4D variant marker” refer to any of the PDE4D splice variants of the human phosphodiesterase PDE4D, i.e., the human phosphodiesterase PDE4D gene, for example PDE4D1, PDE4D2, PDE4D3, PDE4D4, PDE4D5, PDE4D6, PDE4D7, PDE4D8 and PDE4D9.

The term “phosphodiesterase 4D1” or “PDE4D1” relates to the splice variant 1 of the human phosphodiesterase PDE4D, i.e., the human phosphodiesterase PDE4D1 gene, for example, to the sequence as defined in NCBI Reference Sequence: NM_001197222.1, specifically, to the nucleotide sequence as set forth in SEQ ID NO:1, which corresponds to the sequence of the above indicated NCBI Reference Sequence of the PDE4D1 transcript, and also relates to the corresponding amino acid sequence for example as set forth in SEQ ID NO:2, which corresponds to the protein sequence defined in NCBI Protein Accession Reference Sequence NP_001184151.1 encoding the PDE4D1 polypeptide. The term “phosphodiesterase 4D1” or “PDE4D1” also relates to the amplicon that can be generated by the primer pair PDE1D1D2_forward (SEQ ID NO:3) and the PDE1D1D2_reverse (SEQ ID NO:4) and can be detected by probe SEQ ID NO:5.

The term “phosphodiesterase 4D2” or “PDE4D2” refers to the splice variant 2 of the human phosphodiesterase PDE4D, i.e., the human phosphodiesterase PDE4D2 gene, for example, to the sequence as defined in NCBI Reference Sequence: NM_001197221.1, specifically, to the nucleotide sequence as set forth in SEQ ID NO:6, which corresponds to the sequence of the above indicated NCBI Reference Sequence of the PDE4D2 transcript, and also relates to the corresponding amino acid sequence for example as set forth in SEQ ID NO:7, which corresponds to the protein sequence defined in NCBI Protein Accession Reference Sequence NP_001184150.1 encoding the PDE4D2 polypeptide.

The term “phosphodiesterase 4D3” or “PDE4D3” refers to the splice variant 3 of the human phosphodiesterase PDE4D, i.e., the human phosphodiesterase PDE4D3 gene, for example, to the sequence as defined in NCBI Reference Sequence: NM_006203.4, specifically, to the nucleotide sequence as set forth in SEQ ID NO:8, which corresponds to the sequence of the above indicated NCBI Reference Sequence of the PDE4D3 transcript, and also relates to the corresponding amino acid sequence for example as set forth in SEQ ID NO:9, which corresponds to the protein sequence defined in NCBI Protein Accession Reference Sequence NP_006194.2 encoding the PDE4D3 polypeptide.

The term “phosphodiesterase 4D4” or “PDE4D4” refers to the splice variant 4 of the human phosphodiesterase PDE4D, i.e., the human phosphodiesterase PDE4D4 gene, for example, to the sequence as defined in NCBI Reference Sequence: NM_001104631.1, specifically, to the nucleotide sequence as set forth in SEQ ID NO:10, which corresponds to the sequence of the above indicated NCBI Reference Sequence of the PDE4D4 transcript, and also relates to the corresponding amino acid sequence for example as set forth in SEQ ID NO:11, which corresponds to the protein sequence defined in NCBI Protein Accession Reference Sequence NP_001098101.1 encoding the PDE4D4 polypeptide.

The term “phosphodiesterase 4D5” or “PDE4D5” refers to the splice variant 5 of the human phosphodiesterase PDE4D, i.e., the human phosphodiesterase PDE4D5 gene, for example, to the sequence as defined in NCBI Reference Sequence: NM_001197218.1, specifically, to the nucleotide sequence as set forth in SEQ ID NO:12, which corresponds to the sequence of the above indicated NCBI Reference Sequence of the PDE4D5 transcript, and also relates to the corresponding amino acid sequence for example as set forth in SEQ ID NO:13, which corresponds to the protein sequence defined in NCBI Protein Accession Reference Sequence NP_001184147.1 encoding the PDE4D5 polypeptide. The term “phosphodiesterase 4D5” or “PDE4D5” also relates to the amplicon that can be generated by the primer pair PDE4D5_forward (SEQ ID NO:14) and the PDE4D5_reverse (SEQ ID NO:15) and can be detected by probe SEQ ID NO:16.

The term “phosphodiesterase 4D6” or “PDE4D6” refers to the splice variant 6 of the human phosphodiesterase PDE4D, i.e., the human phosphodiesterase PDE4D6 gene, for example, to the sequence as defined in NCBI Reference Sequence: NM_001197223.1, specifically, to the nucleotide sequence as set forth in SEQ ID NO:17, which corresponds to the sequence of the above indicated NCBI Reference Sequence of the PDE4D6 transcript, and also relates to the corresponding amino acid sequence for example as set forth in SEQ ID NO:18, which corresponds to the protein sequence defined in NCBI Protein Accession Reference Sequence NP_001184152.1 encoding the PDE4D6 polypeptide.

The term “phosphodiesterase 4D7” or “PDE4D7” refers to the splice variant 7 of the human phosphodiesterase PDE4D, i.e., the human phosphodiesterase PDE4D7 gene, for example, to the sequence as defined in NCBI Reference Sequence: NM_001165899.1, specifically, to the nucleotide sequence as set forth in SEQ ID NO:19, which corresponds to the sequence of the above indicated NCBI Reference Sequence of the PDE4D7 transcript, and also relates to the corresponding amino acid sequence for example as set forth in SEQ ID NO:20, which corresponds to the protein sequence defined in NCBI Protein Accession Reference Sequence NP_001159371.1 encoding the PDE4D7 polypeptide. The term “phosphodiesterase 4D7” or “PDE4D7” also relates to the amplicon that can be generated by the primer pair PDE4D7_forward (SEQ ID NO:21) and the PDE4D7_reverse (SEQ ID NO:22) and can be detected by probe SEQ ID NO:23.

The PDE4D7 polypeptide can also be detected with primer pair PDE4D7-2_forward (SEQ ID NO:24) and the PDE4D7_reverse (SEQ ID NO:25) and can be detected by probe SEQ ID NO:26.

The term “phosphodiesterase 4D8” or “PDE4D8” relates to the splice variant 8 of the human phosphodiesterase PDE4D, i.e., the human phosphodiesterase PDE4D8 gene, for example, to the sequence as defined in NCBI Reference Sequence: NM_001197219.1, specifically, to the nucleotide sequence as set forth in SEQ ID NO:27, which corresponds to the sequence of the above indicated NCBI Reference Sequence of the PDE4D8 transcript, and also relates to the corresponding amino acid sequence for example as set forth in SEQ ID NO:28, which corresponds to the protein sequence defined in NCBI Protein Accession Reference Sequence NP_001184148.1 encoding the PDE4D8 polypeptide.

The term “phosphodiesterase 4D9” or “PDE4D9” relates to the splice variant 9 of the human phosphodiesterase PDE4D, i.e., the human phosphodiesterase PDE4D9 gene, for example, to the sequence as defined in NCBI Reference Sequence: NM_001197220.1, specifically, to the nucleotide sequence as set forth in SEQ ID NO:29, which corresponds to the sequence of the above indicated NCBI Reference Sequence of the PDE4D9 transcript, and also relates to the corresponding amino acid sequence for example as set forth in SEQ ID NO:30 which corresponds to the protein sequence defined in NCBI Protein Accession Reference Sequence NP_001184149.1 encoding the PDE4D9 polypeptide. The term “phosphodiesterase 4D9” or “PDE4D9” also relates to the amplicon that can be generated by the primer pair PDE4D9_forward (SEQ ID NO:31) and the PDE4D9_reverse (SEQ ID NO:32) and can be detected by probe SEQ ID NO:33.

The terms “PDE4D1,” “PDE4D2,” “PDE4D3,” “PDE4D4,” “PDE4D5,” “PDE4D6,” “PDE4D7,” “PDE4D8” and “PDE4D9” also comprises nucleotide sequences showing a high degree of homology to PDE4D1, PDE4D2, PDE4D3, PDE4D4, PDE4D5, PDE4D6, PDE4D7, PDE4D8 and PDE4D9 respectively, e.g., nucleic acid sequences being at least 75%, 80%, 85%, 90%, 91%, 92%, 93%, 94%, 95%, 96%, 97%, 98% or 99% identical to the sequence as set forth in SEQ ID NOs: 1, 6, 8, 10, 12, 17, 19, 27 or 29 respectively or amino acid sequences being at least 75%, 80%, 85%, 90%, 91%, 92%, 93%, 94%, 95%, 96%, 97%, 98% or 99% identical to the sequence as set forth in SEQ ID NO:2, 7, 9, 11, 13, 18, 20, 28 or 30 respectively or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:2, 7, 9, 11, 13, 18, 20, 28 or 30 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:1, 6, 8, 10, 12, 17, 19, 27 or 29.

The term “expression level” as used herein refers to the amount of PDE4D variant transcript and/or PDE4D protein derivable from a defined number of cells or a defined tissue portion, in particular, to the amount of PDE4D variant transcript and/or PDE4D variant protein obtainable in a standard nucleic acid (e.g., RNA) or protein extraction procedure. Suitable extraction methods are known to the person skilled in the art.

The term “control level” (or “control state”), as used herein, refers to an expression level which may be determined at the same time and/or under similar or comparable conditions as the test sample by using (a) sample(s) previously collected and stored from a subject/subjects whose condition or disease state, e.g., non-cancerous, normal or benign prostate tumor, advanced prostate cancer etc. is/are known. The term “disease state” or “cancerous disease state” relates to any state or type of cellular or molecular condition between a non-cancerous cell state and (including) a terminal cancerous cell state. In particular, the term includes different cancerous proliferation/developmental stages or levels of tumor development in the organism between (and excluding) a non-cancerous cell state and (including) a terminal cancerous cell state. Such developmental stages may include all stages of the TNM (Tumor, Node, Metastasis) classification system of malignant tumors as defined by the UICC, e.g., stages 0 and I to IV. The term also includes stages before TNM stage 0, e.g., developmental stages in which cancer biomarkers known to the person skilled in the art show a modified expression or expression pattern.

The expression level as mentioned above may be the expression level of PDE4D variants as defined herein above. Alternatively or additionally, the expression level may also be the expression level of any other suitable gene or genetic element expressed in a cell e.g., the expression level of a reference gene or the expression level of a combination of reference genes, e.g., one or more of Homo sapiens hypoxanthine phosphoribosyltransferase 1 (HPRT1), Tubulin-Alpha-1b (TUBA1B), Homo sapiens pumilio RNA-Binding Family Member (PUM1), and Homo sapiens TATA box binding protein (TBP). In one embodiment, the expression level is determined for a combination of reference genes.

The term “cancerous” refers to a cancerous disease state as defined herein. The term “non-cancerous” refers to a condition in which neither benign nor malign proliferation can be detected. Suitable means for the detection are known in the art.

The term “prostate cancer” refers to a cancer of the prostate gland in the male reproductive system, which occurs when cells of the prostate mutate and begin to multiply out of control. Typically, prostate cancer is linked to an elevated level of prostate-specific antigen (PSA). In one embodiment of the present invention the term “prostate cancer” relates to a cancer showing PSA levels above 4.0. In another embodiment the term relates to cancer showing PSA levels above 2.0. The term “PSA level” refers to the concentration of PSA in the blood in ng/ml.

The term “non-progressive prostate cancer state” means that a sample of an individual does not show parameter values indicating “biochemical recurrence” and/or “clinical recurrence.”

The term “progressive prostate cancer state” means that a sample of an individual shows parameter values indicating “biochemical recurrence” and/or “clinical recurrence”.

The term “biochemical recurrence” generally refers to recurrent biological values of increased PSA indicating the presence of prostate cancer cells in a sample.

However, it is also possible to use other markers that can be used in the detection of the presence or that rise suspicion of such presence.

The term “clinical recurrence” refers to the presence of clinical signs indicating the presence of tumor cells as measured, for example using in vivo imaging.

The term “increased” or “increased expression level” or “up-regulated expression level” or “increase of expression level” (which may be used synonymously) denotes a raise in the expression level between a situation to be analyzed, e.g., a situation derivable from a patient's sample, and a reference point, which could either be a normal control level or cancerous control level derivable from any suitable prostate tumor or cancer stage known to the person skilled in the art. Expression levels are deemed to be “increased” when the PDE4D variant gene expression, e.g., in a biological sample to be analyzed, differs by, i.e., is elevated by, for example, 5%, 6%, 7%, 8%, 9%, 10%, 15%, 20%, 25%, 30%, 40%, 50%, or more than 50% in comparison to a control level, or by at least 0.1 fold, at least 0.2 fold, at least 1 fold, at least 2 fold, at least 5 fold, or at least 10 fold or more in comparison to a control level. The control level may either be a normal control level or a cancerous control level as defined herein above. If a comparison with a cancerous control level is to be carried out, an additional comparison with a normal control level is preferred. Such an additional comparison allows for the determination of a tendency of the modification, e.g., the magnitude of an increase of the expression level may be observed and/or corresponding conclusions may be drawn. It can be a comparison to a benign prostate tumor, or to a healthy tissue or a sample derived from a healthy individual.

The term “monitoring prostate cancer,” as used herein relates to the accompaniment of a diagnosed or detected prostate cancer disease or disorder, e.g., during a treatment procedure or during a certain period of time, typically during 2 months, 3 months, 4 months, 6 months, 1 year, 2 years, 3 years, 5 years, 10 years, or any other period of time. The term “accompaniment” means that states of disease as defined herein above and, in particular, changes of these states of disease may be detected by comparing the expression level of the PDE4D variant marker in a sample to a normal control level as defined herein above, in particular, a control expression level derived from a progressive tumor control, a non-progressive tumor control or a healthy control or to the expression level of an established, e.g., independently established, prostate cancer cell or cell line, or a cell line in any type of periodical time segment, e.g., every week, every 2 weeks, every month, every 2, 3, 4, 5, 6, 7, 8, 9, 10, 11 or 12 month, every 1.5 year, every 2, 3, 4, 5, 6, 7, 8, 9 or 10 years, during any period of time, e.g., during 2 weeks, 3 weeks, 1, 2, 3, 4, 5, 6, 7, 8, 9, 10, 11, 12 months, 1.5, 2, 3, 4, 5, 6, 7, 8, 9, 10, 15 or 20 years, respectively. The established, e.g., independently established, prostate cancer cell or cell line giving rise to an additional control level may be derived from samples corresponding to different stages of cancer development, e.g., stages 0 and I to IV of the TNM classification system. In one embodiment, the term relates to the accompaniment of a diagnosed prostate cancer, in particular, of a progressive or non-progressive prostate cancer. The monitoring may also include the detection of the expression of additional genes or genetic elements, e.g., reference genes.

The term “prognosticating prostate cancer” as used herein refers to the prediction of the course or outcome of a diagnosed or detected prostate cancer, e.g., during a certain period of time, during a treatment or after a treatment. The term also refers to a determination of chance of survival or recovery from the disease, as well as to a prediction of the expected survival time of a subject. A prognosis may, specifically, involve establishing the likelihood for survival of a subject during a period of time into the future, such as 6 months, 1 year, 2 years, 3 years, 5 years, 10 years or any other period of time.

The terms “diagnosing” and “prognosticating” are also intended to encompass predictions and likelihood analyses. PDE4D variants as markers may accordingly be used clinically in making decisions concerning treatment modalities, including therapeutic intervention or diagnostic criteria such as a surveillance for the disease. According to the present invention, an intermediate result for examining the condition of a subject may be provided. Such intermediate result may be combined with additional information to assist a doctor, nurse, or other practitioner to diagnose that a subject suffers from the disease. Alternatively, the present invention may be used to detect cancerous cells in a subject-derived tissue, and provide a doctor with useful information to diagnose that the subject suffers from the disease.

The term “reference gene” or “control gene” as used herein refers to any suitable gene, e.g., to any steadily expressed and continuously detectable gene, gene product, expression product, protein or protein variant in the organism of choice. The term also includes gene products such as expressed proteins, peptides, polypeptides, as well as modified variants thereof. The term reference gene hence also includes reference proteins derived from a reference gene, unless otherwise noted. Also encompassed are all kinds of transcripts derivable from the reference gene as well as modifications thereof or secondary parameters linked thereto. Alternatively or additionally, other reference parameters may also be used for reference purposes, e.g., metabolic concentrations, cell sizes etc.

The expression may be carried out in the same sample, i.e., the level of a PDE4D variant and of the reference gene is determined in the same sample. If the testing is carried out in the same sample, a single detection or a multiplex detection approach as described herein may be performed. For the performance of the multiplex detection the concentration of primers and/or probe oligonucleotides may be modified. Furthermore, the concentration and presence of further ingredients like buffers, ions etc. may be modified, e.g., increased or decreased in comparison to manufacturers' indications.

In a specific embodiment, the expression of more than one reference gene or steadily expressed gene may be determined. For example, the expression of 2, 3, 4, 5, 6, 7, 8, 9, 10, 12, 15, 20, 30 or more reference genes may be determined. The results of such measurements may be either calculated separately, or may be combined in order to obtain an average expression index. Furthermore, pattern of reference gene expression may be determined and/or used as basis for subsequent steps. Such pattern may be based on known expression behaviors of genes in certain cancer, in particular prostate cancer stages or states.

A subject, such as a patient or individual to be diagnosed, monitored or prognosticated prostate cancer or the progression state of prostate cancer is an animal, such as a mammal, e.g., a human being.

The level of the PDE4D variant may be determined on the nucleic acid level, protein level or activity level as described herein. Preferred is the determination of the amount of PDE4D variant transcript(s) and/or protein. In addition the level of a reference gene in sample may be determined.

In one embodiment, the diagnosing, monitoring, prognosticating, stratifying risk, and providing a recommendation as mentioned herein is to be carried out on a biological sample obtained from an individual. The term “biological sample” or “sample obtained from an individual” refers to any biological material obtained via suitable methods known to the person skilled in the art from an individual. The biological sample used may be collected in a clinically acceptable manner, e.g., in a way that nucleic acids (in particular RNA) or proteins are preserved.

The biological sample(s) may include body tissue and/or a fluid, such as, but not limited to, blood, sweat, and urine. Furthermore, the biological sample may contain a cell extract derived from or a cell population including an epithelial cell, such as a cancerous epithelial cell or an epithelial cell derived from tissue suspected to be cancerous. The biological sample may contain a cell population derived from a glandular tissue, e.g., the sample may be derived from the prostate of a male individual. Additionally, cells may be purified from obtained body tissues and fluids if necessary, and then used as the biological sample. In some embodiments, the sample is a tissue sample, a urine sample, a urine sediment sample, a blood sample, a saliva sample, a semen sample, a sample including circulating tumor cells, extracellular vesicles, a sample containing prostate secreted exosomes, or cell lines or cancer cell line.

In one embodiment, biopsy or resections samples may be obtained and/or used. Such samples may include cells or cell lysates.

In a specific embodiment, the content of a biological sample may also be submitted to an enrichment step. For instance, a sample may be contacted with ligands specific for the cell membrane or organelles of certain cell types, e.g., prostate cells, functionalized for example with magnetic particles. The material concentrated by the magnetic particles may subsequently be used for detection and analysis steps as described herein above or below.

Furthermore, cells, e.g., tumor cells, may be enriched via filtration processes of fluid or liquid samples, e.g., blood, urine, etc. Such filtration processes may also be combined with enrichment steps based on ligand specific interactions as described herein above.

The management of prostate cancer patients is strongly dependent on risk profiling. The National Comprehensive Cancer Network (NCCN) has defined five risk categories (very low risk, VLR; low risk, LR; favorable intermediate risk, FIR; unfavorable intermediate risk, UIR; and high risk, HR), based on pre-treatment parameters, which are illustrated in TABLES 1 and 2.

TABLE 1
Clinical risk stratification for prostate cancer
patients as outlined in the US NCCN guidelines

| | | | | % Positive | | # Positive | |
|---|---|---|---|---|---|---|---|
| | Biopsy | Clinical | | Biopsy | | Biopsy | % Tumor |
| NCCN | Gleason | Stage | PSA | Cores | PSAD | Cores | in Biopsy |
| VLR | 3 + 3 | cT1c | <10 | N/A | <0.15 | <3 | <50% |

| | | | | | |
|---|---|---|---|---|---|
| LR | 3 + 3 | cT1c | <10 | N/A | N/A |
| | | cT2a | | | |
| FIR | 3 + 3 | cT2b | <10 | | |
| | | cT2c | | | |
| | | cT1c | 10-20 | | |
| | | cT2a | | | |
| | 3 + 4 | cT1c | <10 | <50% | N/A |
| | | cT2a | | | |

| | | | | |
|---|---|---|---|---|
| UIR | 3 + 3 | cT2b | 10-20 | N/A |
| | | cT2c | | |
| | 3 + 4 | cT2b | <10 | N/A |
| | | cT2c | | |
| | | cT1c | 10-20 | |
| | | cT2a | | |
| | | cT2b | 10-20 | |
| | | cT2c | | |
| | 4 + 3 | ≤cT2c | ≤20 | N/A |
| HR | ≥4 + 4 | ≥cT3a | >20 | N/A |

TABLE 2
Parameters for risk assignment

| | LR | IR | HR |
|---|---|---|---|
| Biopsy Gleason | 6 | 7 | 8-10 |
| Clinical Stage | cT1, cT2a | cT2b, cT2c | >cT3a |
| PSA | <10 | 10-20 | >20 |

For each risk group ranging from very low, low, intermediate, high, and very high risk, several options of interventions are presented in the guidelines. Although this patient risk assessment is easy to perform and is based on generally available clinical data, its simplicity also contributes to its main disadvantage, which is in the categorization of patients into non-overlapping groups rather than an individual risk per patient irrespective of the clinical risk grouping. As a consequence, a recommended treatment might be ideal for one patient, but might not be suitable for another patient in the same clinical risk group. Thus, one aspect of this invention is to use molecular markers like PDE4D7 to add orthogonal and independent information to the clinical risk description for more stratified therapy selection.

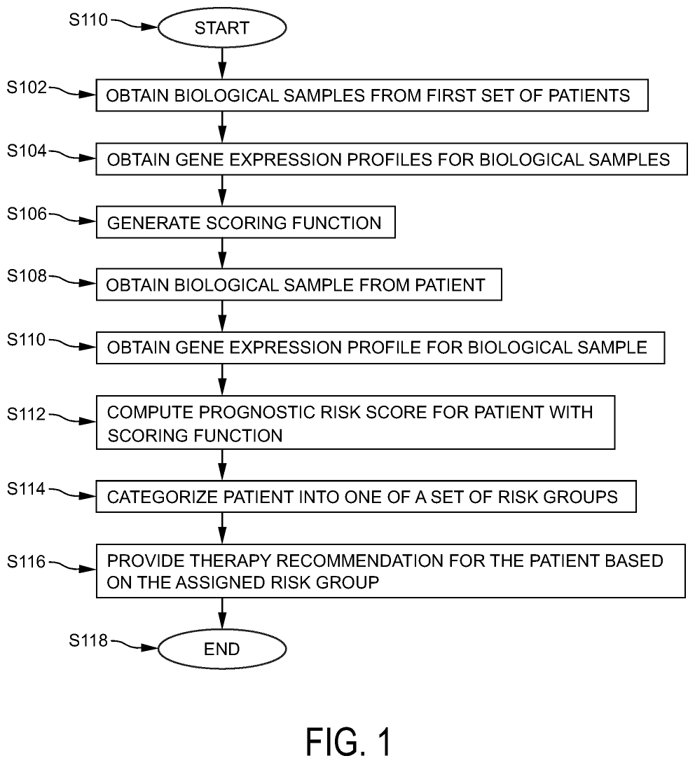

With reference to FIG. 1, a method of risk stratification for therapy selection in a patient with prostate cancer is illustrated. The method begins at S100.

At S102, a biological sample is obtained from each of a first set of patients (individuals) diagnosed with prostate cancer, for whom monitoring prostate cancer has been performed over a period of time, such as at least one year, or at least two years, or about five years, after obtaining the biological sample.

At S104, a gene expression profile for at least one marker gene (e.g., PDE4D7) is obtained for each of the biological samples obtained from the first set of patients, e.g., by performing RT-qPCR (real-time quantitative PCR) on RNA extracted from each biological sample. The exemplary expression profile includes an expression level (e.g., value) for PDE4D7 which can be normalized using value(s) for each of a set of reference genes, such as HPRT1, TUBA1B, PUM1, and/or TBP. In one embodiment, the only marker gene used is PDE4D7 and the only reference genes used are selected from the group consisting of HPRT1, TUBA1B, PUM1, and TBP, e.g., at least one or at least two or at least three or all of these reference genes.

At S106 a scoring function for assigning a prognostic risk score is determined, based on the gene expression profile for the marker gene (PDE4D7) obtained for at least some of the biological samples obtained for the first set of patients and respective results obtained from the monitoring.

At S108, a biological sample is obtained from a patient (individual). The patient can be a new patient or one of the first set.

At S110, a gene expression profile is obtained for the at least one marker gene (e.g., PDE4D7), e.g., by performing PCR on the biological sample. The gene expression profile includes a gene expression level for phosphodiesterase 4D variant 7 (PDE4D7) and for one or more reference genes. Suitable reference genes include HPRT1, TUBA1B, PUM1, and TBP. In one embodiment, the only marker gene used is PDE4D7 and the only reference genes used are selected from the group consisting of HPRT1, TUBA1B, PUM1, and TBP, e.g., at least one or at least two or at least three or all of these reference genes. The marker and reference genes are the same as used in S104.

Other reference genes which may be additionally or alternatively used in steps S104 and S110 include Homo sapiens actin, beta, mRNA (ACTB); Homo sapiens 60S acidic ribosomal phosphoprotein P0 mRNA (RPLP0); Polymerase (RNA) II (DNA Directed) Polypeptide A, 220 kDa (POLR2A); Beta-2-Microglobulin (B2M); and Aminolevulinate-Delta-Synthase (ALAS-1).

At S112, a prognostic risk score is determined for the patient, based on the gene expression profile, using the derived scoring function.

At S114, the patient may be categorized into one of a predefined set of risk groups, based on the prognostic risk score.

At S116, a therapy recommendation may be provided, e.g., to the patient or his or her guardian, to a doctor, or to another healthcare worker, based on the patient's risk group. This may include one or more of a) proposing a therapy for the patient based on the assigned risk group, with at least two of the risk groups being associated with different therapies, b) computing a disease progression risk prediction of the patient before or after prostate surgery; and c) computing a therapy response prediction for the patient before or after prostate surgery. Example therapies include at least a partial prostatectomy, an active therapy selected from radiation treatment, chemotherapy, and a combination thereof, and observation alone, i.e., without performing prostatectomy or active therapy (i.e., active surveillance).

The method ends at S118.

The exemplary scoring function allows new patients to be categorized into a respective one of a set of risk groups to which the first set of patients have been assigned, based on the results of their monitoring. Each of the risk groups may be associated with a respective proposed therapy, which differs in its aggressiveness. Each proposed therapy may be based on the results of the patients from the first set that were assigned to that risk group and is one which is predicted to provide the least aggressive therapy which does not exceed a threshold clinical risk for development of prostate cancer. In some cases, this enables a new patient to be assigned to a risk group associated with a less aggressive proposed therapy than would be the case for other risk profiling methods, such as that using the Gleason score.

In one embodiment, the gene expression level at S104, S110 is determined by detecting mRNA expression using one or more primers and/or probes and/or one or more sets thereof.

In a variant of the method, a combined prognostic risk score is additionally determined for the patient, at S112, based on the prognostic risk score and a second risk determination.

At S114, the patient may be categorized into one of a predefined set of risk groups, based on the combined prognostic risk score (rather than based on the prognostic risk score).

The second risk determination is a risk determination other than the PDE4D7 risk score (prognostic risk score), e.g. it may be based on a Gleason score. Preferably, the second risk determination is a National Comprehensive Cancer Network (NCCN) classification, such as one or more of very low risk (VLR), low risk (LR), favorable intermediate risk (FIR), unfavorable intermediate risk (UIR), and high risk (HR). The combined prognostic risk score may be determined with a regression function derived from subjects that have been monitored for prostate cancer. A further aspect relates to a computer implemented method for diagnosing, monitoring or prognosticating prostate cancer or stratifying the progression risk of prostate cancer, comprising the method steps as described in FIG. 1.

In the context of the present application, the expression “computer implemented method for diagnosing, monitoring or prognosticating prostate cancer or stratifying the progression risk of prostate cancer,” refers to a method wherein software algorithms calculate a risk score and based thereon provide a prognosis for the patient that is analyzed, wherein this method uses raw data obtained upon measurement of the gene expression level of the genes referred to herein and conversion thereof into a risk score using the equation described below.

One or more steps of the method illustrated in FIG. 1 may be implemented in a computer program product that may be executed on a computer. The computer program product may comprise a non-transitory computer-readable recording medium on which a control program is recorded (stored), such as a disk, hard drive, or the like. Common forms of non-transitory computer-readable media include, for example, floppy disks, flexible disks, hard disks, magnetic tape, or any other magnetic storage medium, CD-ROM, DVD, or any other optical medium, a RAM, a PROM, an EPROM, a FLASH-EPROM, or other memory chip or cartridge, or any other non-transitory medium from which a computer can read and use.

Alternatively, the method may be implemented in transitory media, such as a transmittable carrier wave in which the control program is embodied as a data signal using transmission media, such as acoustic or light waves, such as those generated during radio wave and infrared data communications, and the like.

The exemplary method may be implemented on one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphical card CPU (GPU), or PAL, or the like. In general, any device, capable of implementing a finite state machine that is in turn capable of implementing the flowchart shown in FIG. 1, can be used to implement one or more steps of the method of risk stratification for therapy selection in a patient with prostate cancer is illustrated. As will be appreciated, while the steps of the method may all be computer implemented, in some embodiments one or more of the steps may be at least partially performed manually.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

The terms “determining the level of marker gene(s) or GOI's” or “determining the gene expression level” or “determining the expression level of PDE4D variants” refers to the determination of the presence or amount of marker gene(s) or GOI's or PDE4D variant's expression products. The term “level of marker gene(s) or GOI's” thus means the presence or amount of marker gene(s) or GOI's expression products, e.g., transcript(s), and/or the determination of the presence or amount of marker gene(s) or GOI's. The determination of the presence or amount of marker gene(s) or GOI's expression products, may be accomplished by any means known in the art.

The determination of the presence or amount of marker gene(s) or GOI's expression products may be accomplished by the measurement of nucleic acid. Thus, the expression level(s) may be determined by a method involving the detection of an mRNA encoded by the gene.

For example, the measurement of the nucleic acid level of marker gene(s) or GOI's expression may be assessed by purification of nucleic acid molecules (e.g., RNA or cDNA) obtained from the sample, followed by hybridization with specific oligonucleotide probes as defined herein above. Comparison of expression levels may be accomplished visually or by means of an appropriate device. Methods for the detection of mRNA or expression products are known to the person skilled in the art.

Alternatively, the nucleic acid level of marker gene(s) or GOI's expression may be detected in a DNA array or microarray approach. Typically, sample nucleic acids derived from patients to be tested are processed and labeled, e.g., with a fluorescent label. Subsequently, such nucleic acid molecules may be used in a hybridization approach with immobilized capture probes corresponding to the exemplary marker genes. Suitable means for carrying out microarray analyses are known to the person skilled in the art.

In a standard setup a DNA array or microarray comprises immobilized high-density probes to detect a number of genes. The probes on the array are complementary to one or more parts of the sequence of the marker genes. Typically, cDNAs, PCR products, and oligonucleotides are useful as probes.

A DNA array- or microarray-based detection method typically comprises the following steps: (1) Isolating mRNA from a sample and optionally converting the mRNA to cDNA, and subsequently labeling this RNA or cDNA. Methods for isolating RNA, converting it into cDNA and for labeling nucleic acids are described in manuals for micro array technology. (2) Hybridizing the nucleic acids from step 1 with probes for the marker genes. The nucleic acids from a sample can be labeled with a dye, such as the fluorescent dyes Cy3 (red) or Cy5 (blue). Generally a control sample is labeled with a different dye. (3) Detecting the hybridization of the nucleic acids from the sample with the probes and determining at least qualitatively, and more particularly quantitatively, the amounts of mRNA in the sample for marker genes investigated. The difference in the expression level between sample and control can be estimated based on a difference in the signal intensity. These can be measured and analyzed by appropriate software such as, but not limited to the software provided for example by Affymetrix.

There is no limitation on the number of probes corresponding to the marker genes used, which are spotted on a DNA array. Also, a marker gene can be represented by two or more probes, the probes hybridizing to different parts of a gene. Probes are designed for each selected marker gene. Such a probe is typically an oligonucleotide comprising 5-50 nucleotide residues. Longer DNAs can be synthesized by PCR or chemically. Methods for synthesizing such oligonucleotides and applying them on a substrate are well known in the field of micro-arrays. Genes other than the marker genes may be also spotted on the DNA array. For example, a probe for a gene whose expression level is not significantly altered may be spotted on the DNA array to normalize assay results or to compare assay results of multiple arrays or different assays.

In one embodiment, the nucleic acid level of marker gene(s) or GOI's expression may be detected in a quantitative RT-PCR approach, e.g., in a real-time polymerase chain reaction (RT-qPCR) approach following the reverse transcription transcripts of interest. Typically, as first step, a transcript is reverse transcribed into a cDNA molecule according to any suitable method known to the person skilled in the art. A quantitative or real-time PCR approach may subsequently be carried out based on a first DNA strand obtained as described above.

In one embodiment, Taqman or Molecular Beacon probes as principal FRET-based probes of this type may be used for quantitative PCR detection. In both cases, the probes, serve as internal probes which are used in conjunction with a pair of opposing primers that flank the target region of interest, such as a set of marker gene(s) specific oligonucleotides as defined herein above. Upon amplification of a target segment, the probe may selectively bind to the products at an identifying sequence in between the primer sites, thereby causing increases in FRET signaling relative to increases in target frequency.

The Taqman probe to be used for a quantitative PCR approach may include a specific oligonucleotide as defined above of about 22 to 30 bases that is labeled on both ends with a FRET pair. Typically, the 5′ end will have a shorter wavelength fluorophore such as fluorescein (e.g., FAM) and the 3′ end is commonly labeled with a longer wavelength fluorescent quencher (e.g., TAMRA) or a non-fluorescent quencher compound (e.g., Black Hole Quencher). In one embodiment, the probes to be used for quantitative PCR, in particular probes as defined herein above, have no guanine (G) at the 5′ end adjacent to the reporter dye in order to avoid quenching of the reporter fluorescence after the probe is degraded.

A Molecular Beacon probe to be used for a quantitative PCR approach may use FRET interactions to detect and quantify a PCR product, with each probe having a 5′ fluorescent-labeled end and a 3′ quencher-labeled end. This hairpin or stem-loop configuration of the probe structure may include a stem with two short self-binding ends and a loop with a long internal target-specific region of about 20 to 30 bases.

Alternative detection mechanisms which may also be employed in the context of the present invention are directed to a probe fabricated with only a loop structure and without a short complementary stem region. An alternative FRET-based approach for quantitative PCR which may also be used is based on the use of two hybridization probes that bind to adjacent sites on the target wherein the first probe has a fluorescent donor label at the 3′ end and the second probe has a fluorescent acceptor label at its 5′ end.

In a specific embodiment, the gene expression level is determined by an amplification based method and/or microarray analysis and/or RNA sequencing.

The exemplary gene expression profile is a normalized gene expression profile obtained by normalizing the expression level of at least the PDE4D7 variant to the expression of at least one reference gene.

A detailed description of the reference genes including their Transcript ID (NCBI RefSeq) and the corresponding amino acid sequences for the primer pair and probe are shown in TABLE 3. TABLE 3 also shows, for each reference gene, a sense primer, and antisense primer, and a probe sequence that specifically binds to the amplicon.

TABLE 3
Exemplary primer and probe nucleic acid sequences

| | Exemplary | Exemplary | | | |
|---|---|---|---|---|---|
| Gene | NCBI | Protein | | Antisense | |
| Name | RefSeq | Accession | Sense Primer | primer | Probe Sequence |
| PDE4D7 | NM_00116 | NP_00115 | GAACATTCA | TGCCATTG | CTGCCGCTGA |
| | 5899.1 | 9371.1 | ACGACCAAC | TCCACATC | TTGCTATCAC |
| | (SEQ ID | (SEQ ID | CA (SEQ ID | AAAA (SEQ | TTCTGCA (SEQ |
| | NO: 19) | NO: 20) | NO: 21) | ID NO: 22) | ID NO: 23) |
| | | | CGCTGATTG | GTCGTTGA | TTCCCTTGGA |
| | | | CTATCACTT | CTGTGGAC | TCCCATGACC |
| | | | CTGC (SEQ | AAAATTTG | AGCCCATAAG |
| | | | ID NO: 24) | (SEQ ID | GGAA (SEQ ID |
| | | | | NO: 25) | NO: 26) |
| HPRT1 | NM_00019 | NP_00018 | GAGGATTTG | ACAGAGG | ACGTCTTGCT |
| | 4.2 | 5.1 (SEQ | GAAAGGGT | GCTACAAT | CGAGATGTGA |
| | (SEQ ID | ID NO: 35) | GTTTATT | GTGATG | TGAAGG |
| | NO: 34) | | (SEQ ID | (SEQ ID | (SEQ ID NO: 38) |
| | | | NO: 36) | NO: 37) | |
| TUBA1B | NM_00608 | NP_00607 | TGACTCCTT | TGCCAGTG | CCGGGCTGTG |
| | 2.2 (SEQ | 3.2 (SEQ | CAACACCTT | CGAACTTC | TTTGTAGACT |
| | ID NO: 39) | ID NO: 40) | CTTC (SEQ ID | AT (SEQ ID | TGGA (SEQ ID |
| | | | NO: 41) | NO: 42) | NO: 43) |
| PUM1 | NM_00102 | NP_00101 | GCCAGCTTG | CAAAGCC | ATCCACCATG |
| | 0658.1; | 8494.1 | TCTTCAATG | AGCTTCTG | AGTTGGTAGG |
| | (SEQ ID | (SEQ ID | AAAT (SEQ | TTCAAG | CAGC (SEQ ID |
| | NO: 44) | NO: 46); | ID NO: 48) | (SEQ ID | NO: 50) |
| | NM_01467 | NP_05549 | | NO: 49) | |
| | 6.2 (SEQ | 1.1 (SEQ | | | |
| | ID NO: 45) | ID NO: 47) | | | |
| TBP | NM_00319 | NP_00318 | GCCAAGAA | ATAGGGAT | TCAGAACAAC |
| | 4.4 (SEQ | 5.1 (SEQ | GAAAGTGA | TCCGGGAG | AGCCTGCCAC |
| | ID NO: 51) | ID NO: 52) | ACATCAT | TCAT (SEQ | CTTA (SEQ ID |
| | | | (SEQ ID | ID NO: 54) | NO: 55) |
| | | | NO: 53) | | |
| ACTB | NM_00110 | NP_00109 | CCAACCGCG | CCAGAGG | CCATGTACGT |
| | 1.3 SEQ ID | 2.1 (SEQ | AGAAGATG | CGTACAGG | TGCTATCCAG |
| | NO: 56) | ID NO: 57) | A (SEQ ID | GATAG | GCT (SEQ ID |
| | | | NO: 58) | (SEQ ID | NO: 60) |
| | | | | NO: 59) | |
| RPLP0 | NM_00100 | NP_44450 | TAAACCCTG | ACATTTCG | AAGTAGTTGG |
| | 2.3 (SEQ | 5.1/NP_00 | CGTGGCAAT | GATAATCA | ACTTCCAGGT |
| | ID NO: 61) | 0993.1 | (SEQ ID | TCCAATAG | CGCC (SEQ ID |
| | | (SEQ ID | NO: 64) | TTG (SEQ | NO: 66) |
| | | NO: 62/63) | | ID NO: 65) | |
| ALAS-1 | NM_00068 | NP_00067 | AGCCACATC | CGTAGATG | TTTAGCAGCA |
| | 8.5/NM_19 | 9.1/NP_95 | ATCCCTGT | TTATGTCT | TCTGCAACCC |
| | 9166.2 | 4635.1 | (SEQ ID | GCTCAT | GC (SEQ ID |
| | (SEQ ID | (SEQ ID | NO: 71) | (SEQ ID | NO: 73) |
| | NO: 67/68) | NO: 69/70) | | NO: 72) | |

In specific embodiments, the prognostic risk score is based on the normalized gene expression profile that includes the normalized expression level for at least PDE4D7. In some embodiments, none of the PDE4D variants is used as a reference gene. In other words, the PDE4D variant(s) is not used as a reference gene for normalizing the measured expression level. Expression results may be normalized according to any suitable method known to the person skilled in the art. Typically, such tests or corresponding formula, which would be known to the person skilled in the art, would be used to standardize expression data to enable differentiation between real variations in gene expression levels and variations due to the measurement processes. For microarrays, the Robust Multi-array Average (RMA) may be used as normalization approach.

The normalized values may be generated by applying the following:

N(Cq_{gene of interest})=Mean(Cq_{ref gene})−(Cq_{gene of interest}) (1)

where N(Cq_{gene of interest}) is the normalized gene expression value (quantitation cycle, Cq) for the selected gene of interest;

Mean(Cq_{ref gene}) is the arithmetic mean of the PCR Cq values of the reference gene(s); and

Cq_{gene of interest} is the PCR Cq value of the gene of interest.

In particular embodiments, the expression level of the PDE variants and the reference genes were determined by real-time PCR, as described in R. H. D. Böttcher, “Human phosphodiesterase 4D7 (PDE4D7) expression is increased in TMPRSS2-ERG positive primary prostate cancer and independently adds to a reduced risk of post-surgical disease progression,” Br J Cancer, 113, 1502-1511 (2015), herein incorporated (hereinafter “Böttcher 2015”).

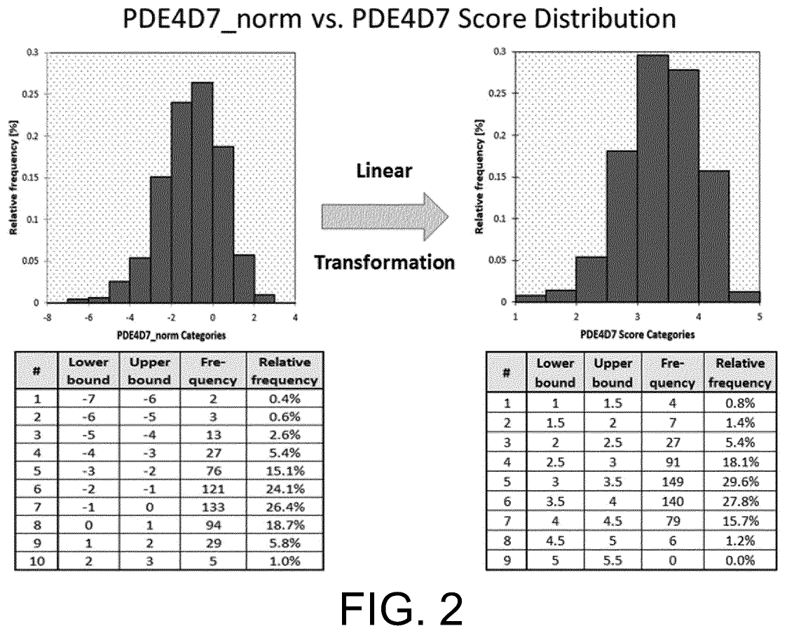

With reference to FIG. 2, in particular embodiments, once the PDE4D7 expression levels are determined and normalized, a prognostic risk score may be determined by applying the following:

PDE4D7 Risk Score=(((PDE4D7_norm+A)*B)+1), (2)

where “PDE4D7 Risk Score” is the prognostic risk score based on the gene expression profile of a sample from a patient, PDE4D7_norm is the normalized PDE4D7 expression value (i.e., N(Cq_{gene of interest})), and A and B are variables.

In particular embodiments, A may be about 6-8, such as 6.7167499999999, B may be 0.4-0.45, such as 0.420780231744713. The PDE4D7 risk score may thus be a value between 1.0 and 5.0. The PDE4D7 risk score can then be classified or categorized into one of at least two risk groups, based on the PDE4D7 risk score. For example, there may be two risk groups, or three risk groups, or four risk groups, or more than four predefined risk groups. Each risk group covers a respective range of (non-overlapping) PDE4D7 risk scores. For example, a risk group may include all PDE4D7 risk scores from 1.0 to 2.0, another risk group from 2.0 to 3.0, another risk group from 3.0 to 4.0, another risk group from 4.0 to 5.0.

In some embodiments, the proposed therapy may be based on the prognostic risk score and on a second risk determination. For example, the second risk determination may be a Gleason score determined by histopathology. See, for example, Sperling, “Revisions of the Gleason grading system make it more accurate,” Sperling Prostate Center, 2016. The second risk determination may also be a clinically defined progression stage (cT value), a pathologically define stage (pT value), a biopsy Gleason score or grouping, a pathology Gleason score or grouping, a prostate-specific antigen measurement, a prostate specific antigen density measurement, or combination thereof.

The second risk determination may be a combination of different risk determinations other than the PDE4D7 risk score. For example, the second risk determination may be an NCCN classification, such as one of very low risk (VLR), low risk (LR), favorable intermediate risk (FIR), unfavorable intermediate risk (UIR), and high risk (HR).

In particular embodiments, the proposed therapy based on the assigned PDE4D7 risk group is different from a potential proposed therapy based only on the second risk determination. That is, the proposed therapy based on the assigned PDE4D7 risk group is different from the proposed therapy based on the second risk determination without the PDE4D7 risk group.

In further embodiments, the PDE4D7 risk group determination stratifies the results and the recommended therapies based on the second risk determination. In other words, the PDE4D7 risk score may identify a patient as not requiring active intervention (i.e., active treatment), and may be placed on active surveillance instead, whereas the second risk determination alone would indicate that active intervention was necessary. Alternatively, the PDE4D7 risk score may identify a patient as requiring active intervention rather than active surveillance whereas the second risk determination alone would indicate that active intervention was not yet necessary.

A further aspect relates to a product including primers and/or probes for determining the expression level of at least one phosphodiesterase 4D (PDE4D) variant selected from the group consisting of PDE4D7, PDE4D1, PDE4D2, PDE4D3, PDE4D4, PDE4D5, PDE4D6, PDE4D8 and PDE4D9 and further comprising primers and/or probes for determining the gene expression level of a reference gene selected from HPRT1, TUBA1B, PUM1, TBP, and combinations thereof. In some embodiments, it is provided with a composition comprising a set of nucleic acid molecules each comprising at least one oligonucleotide primer and/or probe sequence for the analysis of the gene expression of the PDE4D variant(s), and at least one oligonucleotide primer and/or probe sequence for the analysis of the gene expression of reference genes. In some embodiments, it is provided with a nucleic acid array comprising one or more oligonucleotide probes complementary and hybridizable to a coding sequence of the PDE4D variant(s) and one or more oligonucleotide probes complementary and hybridizable to the reference gene(s) for determining a prognostic risk score as defined herein.

A “microarray” is a linear or two-dimensional array of discrete regions, each having a defined area, formed on the surface of a generally solid support such as, but not limited to, glass, plastic, or synthetic membrane. The density of the discrete regions on a microarray is determined by the total numbers of immobilized oligonucleotides to be detected on the surface of a single solid phase support, such as at least about 50/cm², at least about 100/cm², at least about 500/cm², but below about 1,000/cm² in some embodiments. The arrays may contain less than about 500, about 1000, about 1500, about 2000, about 2500, or about 3000 immobilized oligonucleotides in total. As used herein, a DNA microarray is an array of oligonucleotides or oligonucleotides placed on a chip or other surfaces used to hybridize to amplified or cloned oligonucleotides from a sample. Because the position of each particular group of oligonucleotides in the array is known, the identities of a sample oligonucleotides can be determined based on their binding to a particular position in the microarray.

An “oligonucleotide” is a polymeric form of nucleotides, either ribonucleotides or deoxyribonucleotides. This term refers only to the primary structure of the molecule. Thus, this term includes double- and single-stranded DNA and RNA. It also includes known types of modifications including labels known in the art, methylation, “caps,” substitution of one or more of the naturally occurring nucleotides with an analog, and internucleotide modifications such as uncharged linkages (e.g., phosphorothioates, phosphorodithioates, etc.), as well as unmodified forms of the oligonucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. It is possible to further use any sequencing method known in the art to identify the sequences of GOI's.

The term “corresponding” may refer to, where appropriate, a nucleic acid molecule as sharing a substantial amount of sequence identity with another nucleic acid molecule. Substantial amount means at least 95%, usually at least 98% and more usually at least 99%, and sequence identity is determined using the BLAST algorithm, as described in Altschul, et al. J. Mol. Biol. 215:403-410, (1990) (using the published default setting, i.e., parameters w=4, t=17). Methods for amplifying mRNA are generally known in the art, and include reverse transcription PCR (RT-PCR) and those described in U.S. Pat. No. 6,794,141, as well as PCT/US01/50340. Another method which may be used is quantitative PCR (or Q-PCR). Alternatively, RNA may be directly labeled as the corresponding cDNA by methods known in the art.

By relying upon the identification of genes (or expressed sequences) that are over- or under-expressed, one embodiment involves determining expression by hybridization of mRNA, or an amplified or cloned version thereof (such as DNA or cDNA), of a sample cell to a oligonucleotide that is unique to a particular gene sequence. Oligonucleotides of this type may contain at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Other embodiments may use oligonucleotides of at least or about 50, at least or about 100, at least about or 150, at least or about 200, at least or about 250, at least or about 300, at least or about 350, or at least or about 400 basepairs of a gene sequence that is not found in other gene sequences. The term “about” as used in the preceding sentence refers to an increase or decrease of 10% from the stated numerical value. Such oligonucleotides may also be referred to as oligonucleotide probes that are capable of hybridizing to sequences of the genes, or unique portions thereof, described herein. In many cases, the hybridization conditions are stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In other embodiments, oligonucleotide probes useful herein may have about or 95%, about or 96%, about or 97%, about or 98%, or about or 99% identity with the marker gene sequences the expression of which shall be determined. Identity is determined using the BLAST algorithm, as described above. These probes may also be described on the basis of the ability to hybridize to expressed marker genes used in the exemplary method under stringent conditions as described above or conditions equivalent thereto.

In many cases, the sequences are those of mRNA encoded by the marker genes, the corresponding cDNA to such mRNAs, and/or amplified versions of such sequences. In some embodiments, the oligonucleotide probes are immobilized on an array, other devices, or in individual spots that localize the probes.

Suitable labels that can be used according to the invention, include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means. The term “support” refers to conventional supports such as beads, particles, dipsticks, fibers, filters, membranes and silane or silicate supports such as glass slides.

In some embodiments, the product is provided as a kit used to determine a risk score for a subject with localized prostate cancer which includes a) a least one primer and/or probe for determining the expression level of at least one phosphodiesterase 4D (PDE4D) variant, wherein the at least one PDE4D variant comprises PDE4D7, b) at least one primer and/or probe for determining the gene expression level of the at least one reference gene, and c) instructions for computing a risk score based on the determined expressions, e.g., on paper or a disk.

The diagnostic kit may contain one or more agents allowing the specific detection of marker gene(s) or GOI's as defined herein above. The agents or ingredients of a diagnostic kit may be contained in one or more containers or separate entities. The nature of the agents is determined by the method of detection for which the kit is intended.

Furthermore, the kit may include an amount of a known nucleic acid molecule, which can be used for a calibration of the kit or as an internal control. Typically, a diagnostic kit for the detection of marker gene(s) or GOI's expression products may comprise accessory ingredients like a PCR buffers, dNTPs, a polymerase, ions like bivalent cations or monovalent cations, hybridization solutions, etc. Such ingredients are known to the person skilled in the art and may vary depending on the detection method carried out. Additionally, the kit may comprise an instruction leaflet and/or may provide information as to the relevance of the obtained results.

A further aspect relates to a system comprising the above-described products and/or kits and the above-described computer program products. In particular embodiments, the systems, the above-described products and/or kits, and the above-described computer program products may be used in the treatment of prostate cancer.

Without intending to limit the scope of the exemplary embodiment, the following examples illustrate aspects of the method.

### EXAMPLES

### Gene Selection and Cohort Samples Used to Build the PDE4D7 Risk Score for Prostate Cancer

To select gene candidates to build the PDE4D7 risk score, PDE4D7 expression was examined within a cohort of over 500 patients and compared against longitudinal clinically and biologically relevant patient outcomes after primary treatment. A small biopsy punch (approximately 1 millimeter by 2 millimeters) of tissue was collected of a representative tumor area from the resected prostate from 550 patients who had been consecutively operated on between 2000 and 2004. This patient cohort represented a mix of all clinical risk groups according to the definition of, for example, the American NCCN prostate cancer guidelines. After removal of samples which did not meet the pre-defined quality criteria for the biomarker quantification by qPCR and removal of patient who underwent adjuvant hormone therapy after surgery, a total of 503 patient samples were eligible for analysis, as seen in TABLE 4.

TABLE 4
Demographics of the study patient cohort

| | | Total cohort | |
|---|---|---|---|
| Surgery: 2000-2004 | Parameter | (#503) | |
| Clinical | Age | 41.3-74.5 | |
| Range | | (62.6; 7.4) | |
| (median; IQR) | Preoperative PSA | 0.18-73.16 | |
| | | (6.7; 5.5) | |
| | Percent tumor in | 0.2-79.7 | |
| | biopsy | (10.3; 16.0) | |
| | Prostate Volume | 9-148 | |
| | | (42; 22.5) | |
| | PSA density | 0.18-73.2 | |
| | | (6.7; 5.5) | |
| NCCN Risk category | Very Low Risk | 67 | |
| No. of patients | Low Risk | 144 | |
| (percentage) | Favorable | 128 | |
| | Intermediate Risk | | |
| | Unfavorable | 120 | |
| | Intermediate Risk | | |
| | High Risk | 44 | |
| Pre-surgery | Biopsy Gleason 3 + 3 | 316 | (62.8%) |
| pathology | (GG1) | | |
| No. of patients | Biopsy Gleason 3 + 4 | 149 | (29.6%) |
| (percentage) | (GG2) | | |
| | Biopsy Gleason 4 + 3 | 25 | (5.0%) |
| | (GG3) | | |
| | Biopsy Gleason ≥4 + 4 | 13 | (2.6%) |
| | (≥GG4) | | |
| | cT1 | 342 | (68%) |
| | cT2 | 150 | (29.8%) |
| | cT3 | 11 | (2.2%) |
| Post-surgery | Pathology Gleason 3 + 3 | 201 | (40%) |
| pathology | (GG1) | | |
| No. of patients | Pathology Gleason 3 + 4 | 257 | (51.1%) |
| (percentage) | (GG2) | | |
| | Pathology Gleason 4 + 3 | 41 | (8.2%) |
| | (GG3) | | |
| | Pathology Gleason ≥=4 + | 4 | (0.8%) |
| | 4 (>GG4) | | |
| | pT1 | 0 | (0%) |
| | pT2 | 331 | (65.8%) |
| | pT ≥ 3 | 172 | (34.2%) |
| | Positive Surgical | 120 | (23.9%) |
| | Margin | | |
| | Positive Seminal | 60 | (11.9%) |
| | Vesicle Invasion | | |
| | Positive Lymph | 5 | (1%) |
| | Node Invasion | | |

| | | |
|---|---|---|
| Follow-up | Mean | 110.4 |
| Months | IQR median | 120.7 |
| Outcome | <5y BCR | 20.6% |
| Percentage | <10y BCR | 38.6% |
| | <5y CR | 1.1% |
| | <10y CR | 4.3% |
| Salvage Treatment | <5y SRT | 11.8% |
| Percentage | <10y SRT | 25.9% |
| | <5y SADT | 6.1% |
| | <10y SADT | 17.3% |
| Mortality | <5y PCSM | 1.1% |
| Percentage | <10y PCSM | 3.3% |
| | <5y OM | 3.8% |
| | <10y OM | 11.5% |

For patient age, preoperative PSA, percentage of tumor in biopsy, prostate volume, and PSA density, the minimum and maximum values in the cohort are shown, while the median and IQR values are depicted in parentheses. For the NCCN Risk categories, the number of patients per risk group are shown. In case of pre-surgical pathology, the biopsy Gleason grade groups as well as clinical stages are indicated (by number percentage of patients). Post-surgical pathology is represented by the pathology Gleason grade groups, the pathology stages, the surgical margin status after prostatectomy, the tumor invasion status of the seminal vesicles and pelvic lymph nodes (by number percentage of patients).

The follow-up demonstrates the mean and median follow-up periods in months after surgery for all patients. The outcome category illustrates the cumulative 5- and 10-year biochemical recurrence (BCR) and clinical recurrence to metastases (CR) post primary treatment. The treatment lists the 5- and 10-year start to salvage radiation therapy (SRT) or salvage androgen deprivation therapy (SADT) after surgery. Mortality is shown as prostate cancer specific mortality (PCSM) as well as overall mortality (OM).

### Laboratory Methods

The primers and probes used for the quantitative real-time PCR to measure the genes of interest as well as the reference genes are as described in TABLE 3. All molecular biology methods used herein were described previously in Böttcher 2015.

### Data Analysis and Statistics

To enable the comparison of qPCR data across different experiments, the Cq value for PDE4D7 is normalized against the mean of the Cq values for the reference genes to generate a normalized PDE4D7 expression value according to Eqn. (1), where the reference genes used are HPRT1, TUBA1B, PUM1, and TBP.

To determine the correlation of PDE4D7 to clinical outcomes, the normalized PDE4D7 expression was converted to the PDE4D7 risk score by linear transformation (Eqn. 2), as seen in FIG. 2.

Then, the PDE4D7 risk categories were defined by merging all PDE4D7 risk scores between 1 and 2, between 2 and 3, between 3 and 4, and between 4 and 5.

Then, the PDE4D7 risk categories were tested against the various available biological and treatment related outcomes. For statistical analysis, the software package MedCalc was used (MedCalc Software BVBA, Ostend, Belgium).

### Results

With reference to TABLE 5, the differential expression of the PDE4D7 risk score was evaluated in a subset of the patient cohort covering 446 and 347 patients with complete 5-year and 10-year follow-up for biochemical relapse after surgery, respectively. As a reference, two additional prognostic risk scores were determined based on two other PDE4D transcripts, PDE4D5 and PDE4D9, which are also known to be expressed in the prostate.

TABLE 5
Results of a Mann-Whitney U test performed to determine the
differential expression of PDE4D5, PDE4D7, and PDE4D9 in a
patient sub-cohort with complete outcome and follow-up over
5 years (446 patients) or 10 years after surgery (347 patients)

| | PDE4D5 Score | PDE4D7 Score | PDE4D9 Score |
|---|---|---|---|
| Mann-Whitney U Test | (p-value) | (p-value) | (p-value) |
| 5-year BCR (#446/ | 6.30 e−02 | 3.42 e−06 | 6.80 e−01 |
| #92; 18.9%) | | | |
| 10-year BCR (#347/ | 5.50 e−01 | 2.34 e−06 | 9.80 e−01 |
| #134; 38.6%) | | | |
| −PSUPG (#300) | 1.20 e−01 | 7.30 e−01 | 6.30 e−03 |
| vs. +PSUPG (#146) | | | |

As seen in TABLE 5, the PDE4D7 risk score was significantly differently expressed between patients with or without a 5- or 10-year biochemical relapse; however, neither PDE4D5 nor PDE4D9 were able to discriminate between these two subsets of the patient cohort. This demonstrates the unique ability of the PDE4D7 risk score to differentiate between clinical outcomes.

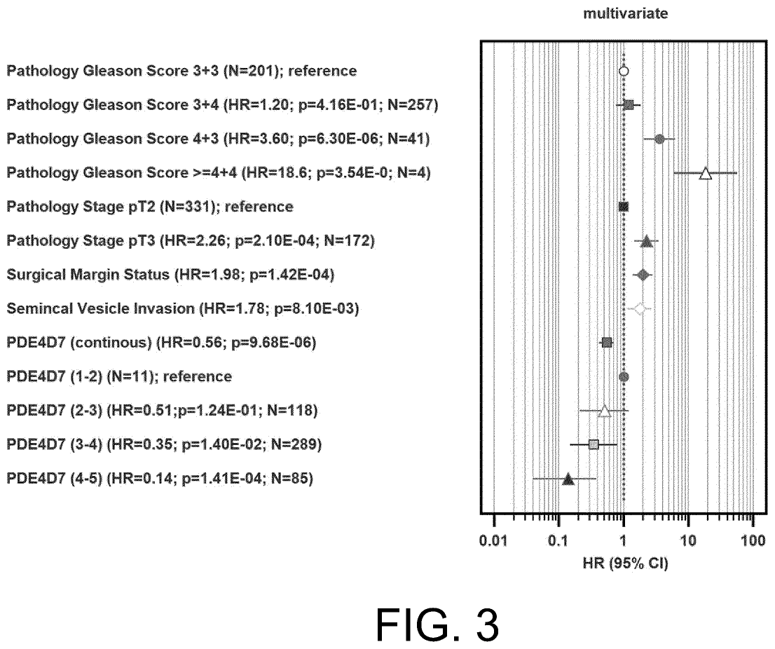

With reference to TABLE 6 and FIG. 3, the univariate and multivariate Cox regression analyses demonstrate a very significant correlation of the continuous PDE4D7 risk score to time to biochemical relapse (BCR) after surgery (HR=0.5; 95% CI=0.4-0.7; p=2.5E−07). Furthermore, when adjusted to known prognostic post-surgical clinical parameters, the PDE4D7 continued to add significant independent value to the regression model (HR=0.5; 95% CI=0.4-0.7; p=9.7E−06). When using the lowest PDE4D7 risk group (i.e., PDE4D7 (1-2)) as a reference, the PDE4D7 risk categories with higher expression levels of PDE4D7 demonstrated a strong decrease in the risk of biochemical relapse over time in the multi-variate analysis as compared to the PDE4D7 reference risk category (PDE4D7 (4-5): HR=0.1; 95% C=0.1-0.5; p=1.4E−04; PDE4D7 (3-4): HR=0.3; 95% CI=0.1-0.8; p=1.4E−02).

TABLE 6
Uni- and multi-variate Cox regression analysis of the continuous and categorized
PDE4D7 risk score in the total patient cohort (503 patients), with the clinical
endpoint of biochemical recurrence, wherein the PDE4D7 risk score was adjusted
by post-surgical clinical parameters in the multi-variate analysis

| Post-Surgical | | | | | | |
|---|---|---|---|---|---|---|
| Clinical | | | | | | |
| parameters | Univariate (enter) | | | Multivariate (enter) | | |
| Endpoint BCR | | | 95% CI | | | 95% CI |
| (#503/#144; 28.6%) | p value | HR | of HR | p value | HR | of HR |

Pathology Gleason Score 3 + 3 N = 201, Reference

| | | | | | | |
|---|---|---|---|---|---|---|
| Pathology Gleason | 1.10 e−03 | 1.96 | 1.31-2.93 | 4.16 e−01 | 1.20 | 0.77-1.87 |
| Score 3 + 4 | | | | | | |
| (N = 257) | | | | | | |
| Pathology Gleason | <1.0 e−14 | 8.28 | 5.02-13.6 | 6.3 e−06 | 3.6 | 2.06-6.28 |
| Score 4 + 3 | | | | | | |
| (N = 41) | | | | | | |
| Pathology Gleason | 1.02 e−09 | 26.4 | 9.22-75.4 | 3.54 e−07 | 18.6 | 6.03-57.1 |
| Score ≥4 + 4 | | | | | | |
| (N = 4) | | | | | | |

Pathology Stage pT2 (N = 331); Reference

| | | | | | | |
|---|---|---|---|---|---|---|
| Pathology Stage | <1.0 e−14 | 4.18 | 2.97-5.86 | 2.10 e−04 | 2.26 | 1.46-3.47 |
| pT3 (N = 172) | | | | | | |
| Surgical Margin | 2.92 e−08 | 2.59 | 1.84-3.62 | 1.42 e−04 | 1.98 | 1.39-2.82 |
| Status (SMS) | | | | | | |
| Seminal Vesicle | <1.0 e−14 | 4.43 | 3.08-6.36 | 8.10 e−03 | 1.78 | 1.16-2.72 |
| Invasion (SVI) | | | | | | |
| PDE4D7 Risk | 2.46 e−07 | 0.52 | 0.41-0.67 | 9.68 e−06 | 0.55 | 0.42-0.72 |
| Score (continuous) | | | | | | |

PDE4D7 Risk (1-2) (N = 11); reference

| | | | | | | |
|---|---|---|---|---|---|---|
| PDE4D7 Risk | 3.50 e−01 | 0.67 | 0.29-1.56 | 1.24 e−01 | 0.51 | 0.21-1.20 |
| (2-3) (N = 117) | | | | | | |
| PDE4D7 Risk | 1.63 e−02 | 0.36 | 0.16-0.83 | 1.40 e−02 | 0.35 | 0.15-0.80 |
| (3-4) (N = 290) | | | | | | |
| PDE4D7 Risk | 5.64 e−04 | 0.18 | 0.07-0.47 | 1.41 e−04 | 0.14 | 0.04-0.38 |
| (4-5) (N = 85) | | | | | | |

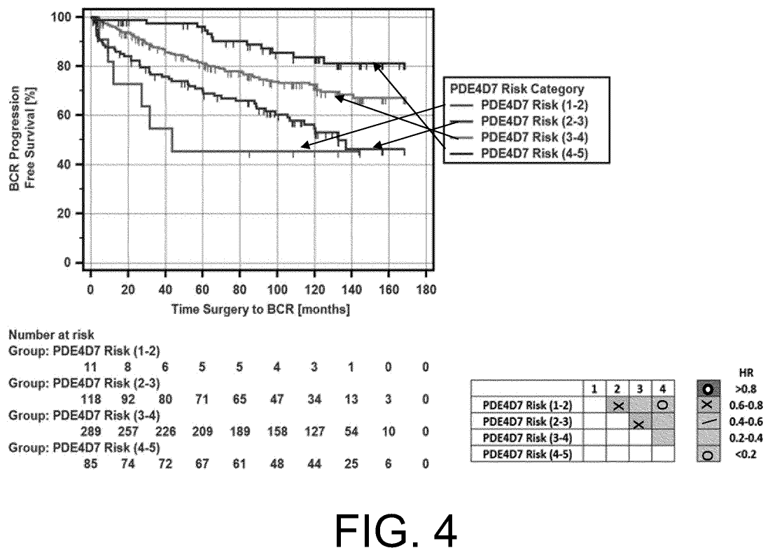

This is confirmed by the Kaplan-Meier analysis performed on the PDE4D7 risk categories with time to PSA recurrence as the clinical endpoint, as seen in FIG. 4. The highest risk category of PDE4D7 includes men with a less than 500 probability of a 5-year BCR, while the chance to experience a PSA recurrence increases to greater than 5000 in the patient group with the lowest levels of PDE4D7 risk score. Notably, all BCR events in the patient cohort with the lowest PDE4D7 risk scores occur within approximately 3.5 years after surgery, while there is no further event after this time period.

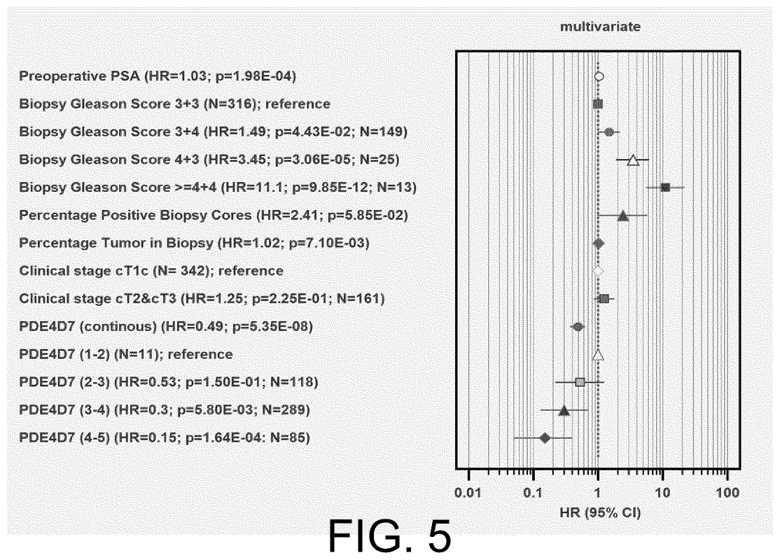

With reference to TABLE 7 and FIG. 5, the independent value of the PDE4D7 risk score in a multivariate analysis when also adjusted to known prognostic pre-surgical clinical parameters was determined. As can be seen, when compared with the multivariate analysis with pre-surgical clinical data for the continuous PDE4D7 risk score, similar results were observed.

TABLE 7
Uni- and multi-variate Cox regression analysis of the continuous and categorized
PDE4D7 risk score in the total patient cohort (503 patients), with the clinical
endpoint of biochemical recurrence, wherein the PDE4D7 risk score was adjusted
by pre-surgical clinical parameters in the multi-variate analysis

| Pre-Surgical | | | | | | |
|---|---|---|---|---|---|---|
| Clinical | | | | | | |
| parameters | Univariate (enter) | | | Multivariate (enter) | | |
| Endpoint BCR | | | 95% CI | | | 95% CI |
| (#503/#144; 28.6%) | p value | HR | of HR | p value | HR | of HR |
| Age at Surgery | 8.01 e−1 | 1.00 | 0.97-1.03 | N/A | N/A | N/A |
| Preoperative PSA | 1.99 e−04 | 1.02 | 1.01-1.03 | 1.88 e−04 | 1.03 | 1.01-1.04 |

Biopsy Gleason Score 3 + 3 N = 316, Reference

| | | | | | | |
|---|---|---|---|---|---|---|
| Biopsy Gleason | 1.30 e−03 | 1.82 | 1.26-2.63 | 4.43 e−02 | 1.49 | 1.01-2.21 |
| Score 3 + 4 | | | | | | |
| (N = 149) | | | | | | |
| Biopsy Gleason | 4.60 e−08 | 4.60 | 2.66-7.95 | 3.02 e−05 | 3.45 | 1.92-6.17 |
| Score 4 + 3 | | | | | | |
| (N = 25) | | | | | | |
| Biopsy Gleason | 4.04 e−13 | 10.9 | 5.7-20.7 | 9.93 e−12 | 11.1 | 5.54-22.1 |
| Score ≥4 + 4 | | | | | | |
| (N = 13) | | | | | | |
| % positive | 1.25 e−06 | 4.51 | 2.45-8.3 | 5.48 e−02 | 2.41 | 0.98-5.92 |
| biopsy cores | | | | | | |
| % tumor in biopsy | 7.03 e−12 | 1.03 | 1.02-1.04 | 7.80 e−03 | 1.02 | 1.00-1.03 |

Clinical Stage cT1c (N = 342); Reference

| | | | | | | |
|---|---|---|---|---|---|---|
| Clinical Stage | 5.26 e−05 | 1.97 | 1.41-2.74 | 2.20 e−01 | 1.25 | 0.87-1.8 |
| cT2 and cT3 | | | | | | |
| (N = 161) | | | | | | |
| PDE4D7 Risk | 2.46 e−07 | 0.52 | 0.41-0.67 | 5.40 e−08 | 0.49 | 0.37-0.63 |
| Score (continuous) | | | | | | |

PDE4D7 Risk (1-2) (N = 11); reference

| | | | | | | |
|---|---|---|---|---|---|---|
| PDE4D7 Risk | 3.50 e−01 | 0.67 | 0.28-1.55 | 1.49 e−01 | 0.53 | 0.22-1.25 |
| (2-3) (N = 117) | | | | | | |
| PDE4D7 Risk | 1.63 e−02 | 0.36 | 0.15-0.82 | 5.80 e−03 | 0.30 | 0.13-0.70 |
| (3-4) (N = 290) | | | | | | |
| PDE4D7 Risk | 5.64 e−04 | 0.18 | 0.06-0.47 | 1.62 e−04 | 0.15 | 0.05-0.40 |
| (4-5) (N = 85) | | | | | | |

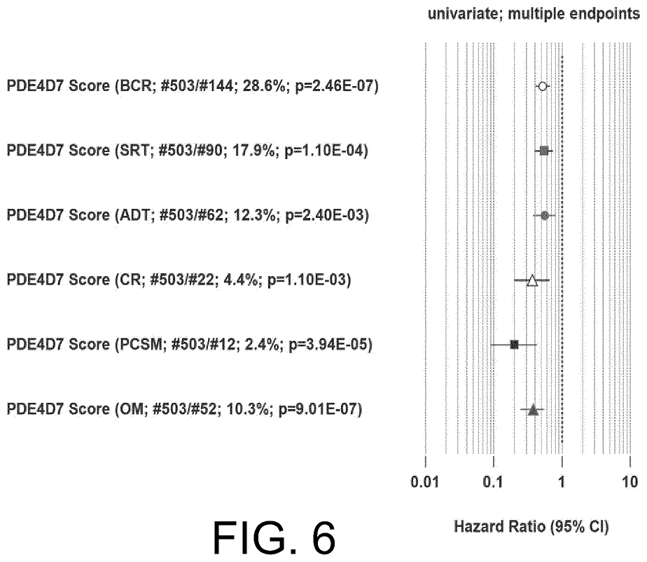

With reference to TABLE 8 and FIG. 6, the correlation of the continuous PDE4D7 risk score in a univariate analysis to time to clinical endpoints after surgery other than biochemical recurrence is shown, with endpoints including: start to salvage radiotherapy (SRI); start to salvage androgen deprivation therapy (SADT); clinical recurrence (CR); prostate cancer specific mortality (PCSM); and overall mortality (OM). As can be seen, the PDE4D7 is significantly negatively correlated to the time point of all endpoints with Hazard ratios between 0.2 and 0.5. Moreover, the likelihood of experiences a serious clinical endpoint like metastases (CR) or death due to prostate cancer (PCSM) increases in particular with decreasing levels of PDE4D7. Additionally, the PDE4D7 risk score appears to have a significant correlation with overall survival.

TABLE 8
Continuous PDE4D7 risk score in a univariate analysis
to time to clinical endpoints after surgery

| Univariate Analysis | Univariate (enter) | | |
|---|---|---|---|
| Mutivariate endpoints | p value | HR | 95% CI of HR |
| PDE4D7 (BCR; #503/ | 2.46 e−07 | 0.52 | 0.41-0.67 |
| #144; 28.6%) | | | |
| PDE4D7 (SRT; #503/ | 1.10 e−04 | 0.55 | 0.40-0.74 |
| #90; 17.9%) | | | |
| PDE4D7 (ADT; #503/ | 2.40 e−03 | 0.56 | 0.38-0.81 |
| #162; 12.3%) | | | |
| PDE4D7 (CR; #503/ | 1.10 e−03 | 0.37 | 0.20-0.66 |
| #22; 4.4%) | | | |
| PDE4D7 (PCSS; #503/ | 3.94 e−05 | 0.20 | 0.09-0.43 |
| #12; 2.4%) | | | |
| PDE4D7 (OS; #503/ | 9.01 e−07 | 0.38 | 0.25-0.55 |
| #52; 10.3%) | | | |

Thus, as can be seen in FIGS. 3, 5, and 6, not only does PDE4D7 expression in tumor tissue have a significant negative correlation to biological outcomes such as BCR, this negative correlation has been shown to provide independent value in multivariate modeling when adjusting to a range of known prognostic pre- and post-surgical clinical parameters. Moreover, PDE4D7 expression levels can also predict other clinical endpoints like the start of salvage treatment as well as endpoints related to disease progression and cancer specific death.

### Stratification—PDE4D7 Risk Score Analysis in Clinically Defined Risk Groups

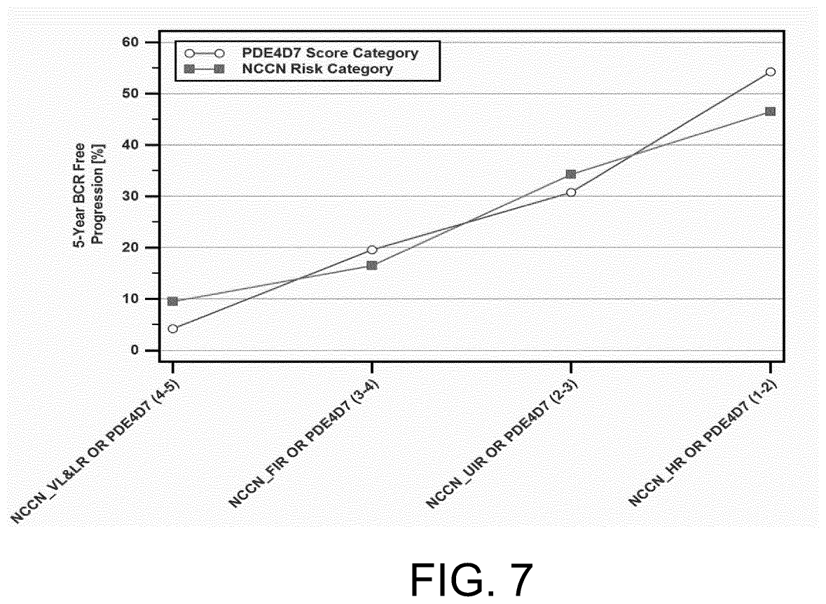

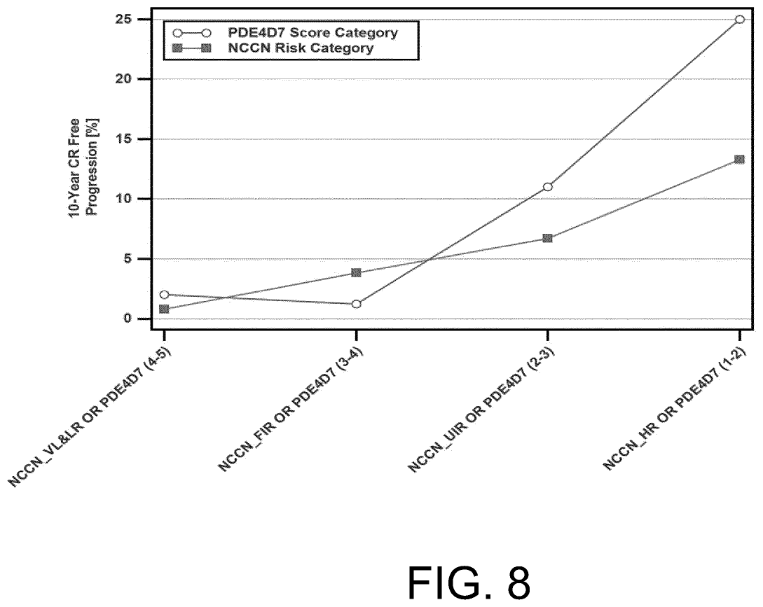

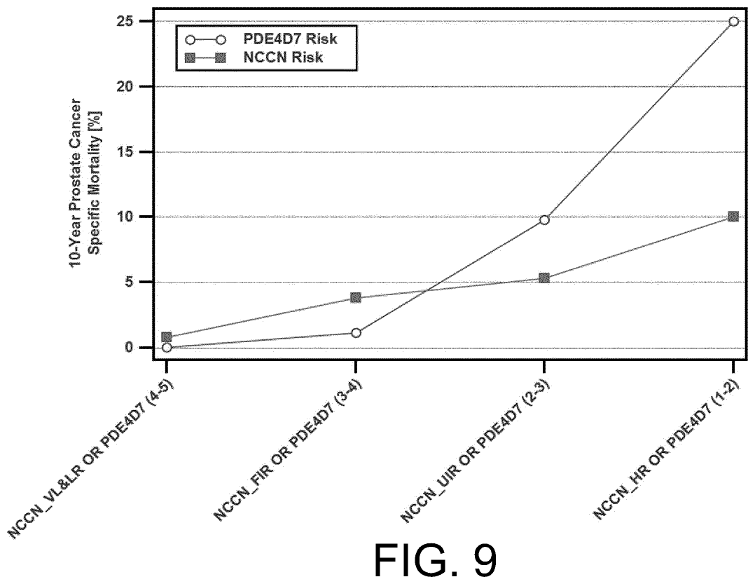

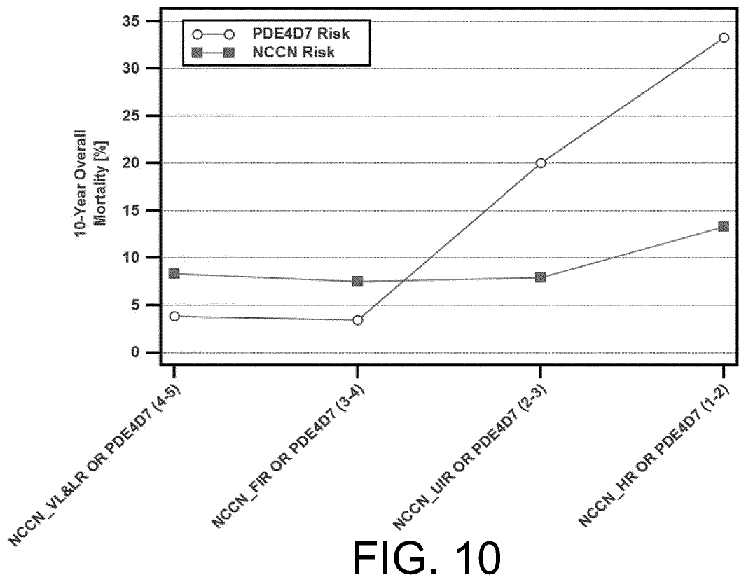

With reference to FIGS. 7-10, the added value of the PDE4D7 risk score on top of clinically defined risk groups as exemplified by the prostate cancer NCCN guideline risk group definitions is illustrated. Four defined PDE4D7 risk groups are compared with the NCCN risk groups for: the 5-year chance to experience the endpoint biochemical recurrence (BCR) after surgery (FIG. 7); the 10-year chance to reach the endpoint clinical recurrence (CR) (FIG. 8); the 10-year chance to reach the endpoint of prostate cancer specific mortality (CSM) (FIG. 9); and the 10-year chance to reach the endpoint of overall mortality (OM) (FIG. 10). The NCCN risk groups included: (1) the very low and low risk group (VL&LR); (2) the favorable intermediate risk group (FIR); (3) the unfavorable intermediate risk group (UIR); and (4) the high risk group (HR). These NCCN risk groups were compared respectively with four PDE4D7 risk groups including: (1) PDE4D7 (4-5); (2) PDE4D7 (3-4); (3) PDE4D7 (2-3); and (4) PDE4D7 (1-2).

As can be seen in FIGS. 7-10, while an increasing risk group contributes to an increased probability in reaching one of the investigated endpoints, the risk distribution by the low risk schemas is different across the four risk categories respectively. This is especially shown in FIGS. 9 and 10, where the increase in risk along the NCCN risk groups is very linear and the slope increase is not very steep. In contrast, there is little 10-year risk of metastases or prostate cancer related death in the two highest PDE4D7 risk categories (3-4 and 4-5), while the slope increase in the groups with lower PDE4D7 risk scores (2-3 and 1-2) strongly increases to reach a final risk level of 25% in the lowest PDE4D7 risk category compared to 10-15% in the NCCN high risk group.

These differences between the NCCN risk groups and the PDE4D7 risk groups can help stratify patients with a prostate cancer diagnosis into, for example, patients that can delay immediate active intervention and instead be treated with active surveillance, and patients that should not be treated with an active intervention therapy. In other words, the PDE4D7 risk score and risk groups can help healthcare providers to recommend to a patient different or alternate therapies based on the additional information provided by the PDE4D7 risk score. That is, the PDE4D7 risk score can help healthcare providers to recommend a patient be placed on active surveillance rather than undergo an active intervention therapy because, based on the PDE4D7, the patient's risk of experiencing one or more particular clinical endpoints is slim or below a particular threshold, even when the patient is classified as being higher risk according to other clinical metrics.

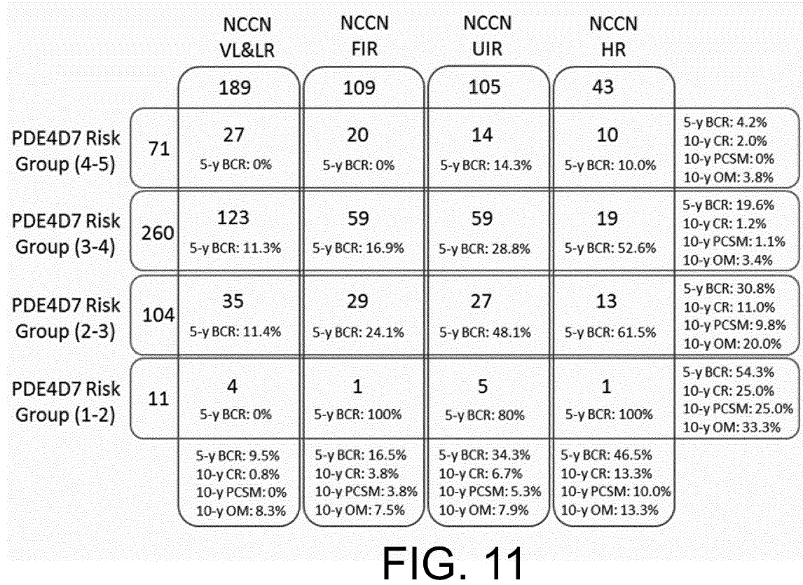

With reference to FIG. 11, the chance of 5-year BCR across all combinations of the four NCCN risk groups versus all PDE4D7 risk categories is illustrated. As expected from the previous analysis, the patient groups representing the highest PDE4D7 risk category (4-5) have less chance to experience one of the measured longitudinal outcomes compared to the NCCN clinical group of very low & low risk, and vice versa for the patient cohort with the lowest PDE4D7 risk (1-2) compared to the NCCN clinically high risk group. Notably, there is a cohort of men defined by high levels of PDE4D7 expression within their tumors who have >50% less risk for BCR within 5 years after surgery compared to the clinical very low and low risk group (4.2% vs. 9.5%, respectively). This is still the case when only considering the clinical very low risk group (4.2% vs. 6.6%, respectively; not shown). Moreover, this high PDE4D7 expressing cohort is composed of men from all clinical risks groups, including the unfavorable intermediate and high risk group.

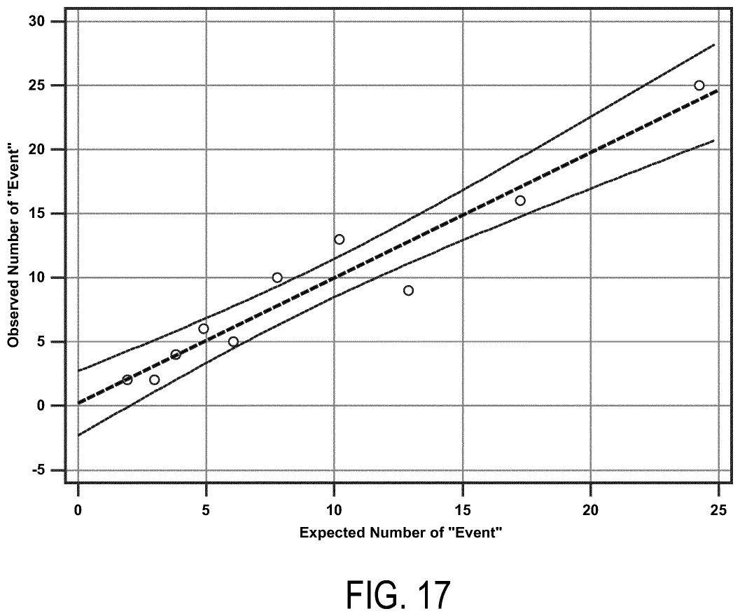

Thus, as can be seen in FIG. 11, the PDE4D7 risk score can be used in combination with a second risk determination, such as the NCCN risk group, to determine a recommended or proposed therapy or treatment. Moreover, the additional information of the PDE4D7 risk score can help stratify patients in order to provide different, alternate, or more appropriate treatments. For example, as seen in FIG. 17, there are 148 patients classified in the NCCN UIR (unfavorable intermediate) and HR (high risk) risk groups. Based on that analysis alone, these patients may elect for immediate active intervention because there is a 34.4% and 46.5% chance of a 5-year BCR. However, by considering the PDE4D7 risk score, these patients may be stratified in a way that differentiates between their actual risk of a 5-year BCR. As a result, the 14 patients with a 14.3% chance of a 5-year BCR, the 10 patients with a 10% chance of a 5-year BCR, and even the 59 patients with a 28.8% chance of a 5-year BCR may instead choose active surveillance rather than an active intervention therapy, thereby delaying the many serious side-effects of active intervention therapies and improving these patients' quality of life. In other words, a healthcare provider may recommend to these 24 or 83 patients undergo active surveillance rather than active intervention, even though the NCCN risk group would suggest that they should undergo some active intervention treatment.

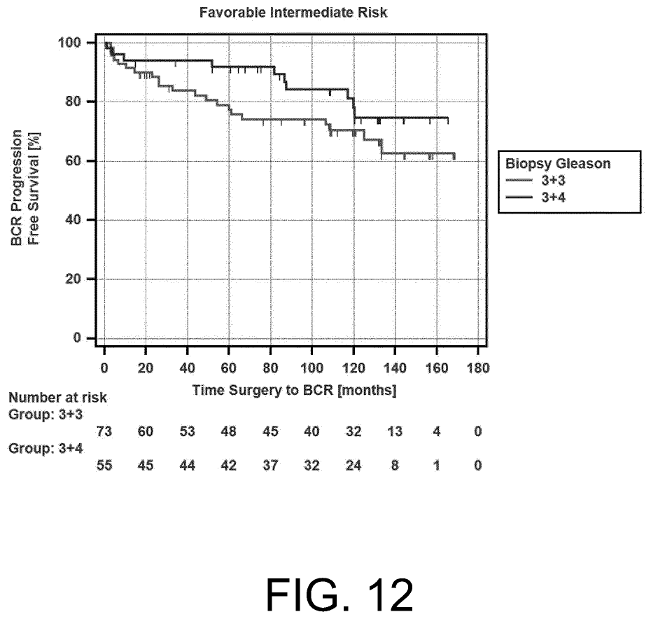

With reference to FIGS. 12-15, the impact of the biopsy Gleason score versus the PDE4D7 risk category in the clinical risk subgroups very low and low risk (VL&LR), favorable intermediate risk (FIR), and unfavorable intermediate risk & high risk (UI&HR) was measured by Kaplan-Meier survival analysis for time to biochemical and clinical relapse. In the case of the VL&L risk group (not shown), there was no significant impact of the PDE4D7 risk categories to stratify the patient sub-cohort further into different risk groups. This may indicate that the overall risk in this group is already very low and consequently it is hard to further sub-stratify this patient cohort. In other words, the clinical low risk group (PSA <10 ng/ml, biopsy Gleason ≤3+3; cT≤T2) is a distinct group with little genomic alterations which harbors little risk of future disease aggressiveness, and which is reflected by a <10% chance of a 5-year biochemical relapse, a <1% chance of a 10-year progression to metastases, and no risk of prostate cancer specific death over 10 years after primary treatment.

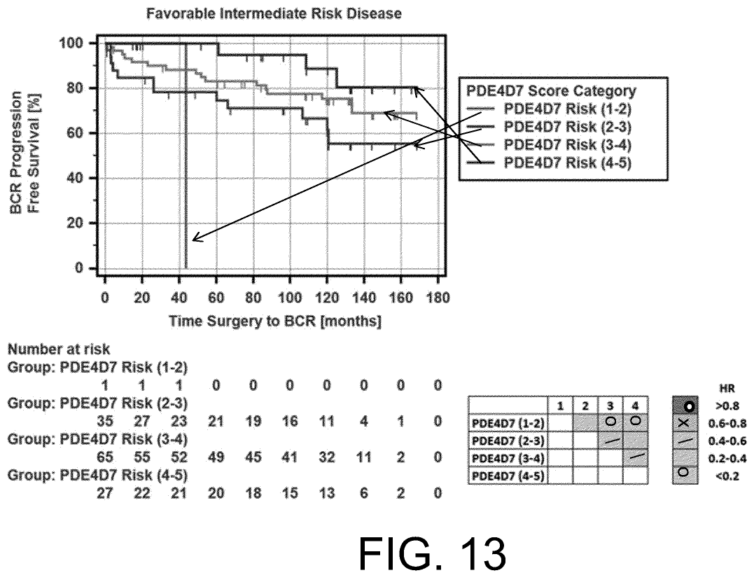

However, as seen in FIGS. 12 and 13, when analyzing the favorable intermediate risk group, it was evident that the biopsy Gleason does not further significantly risk stratify this group (FIG. 12, p=0.19), while the PDE4D7 risk categories clearly define various subsets of patients with different longitudinal risk profiles (FIG. 13, p=0.01).

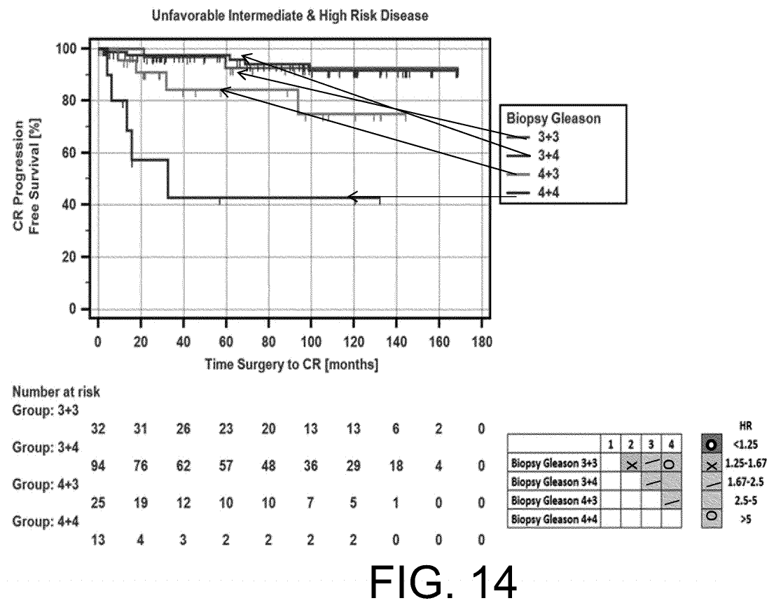

Similarly, as seen in FIG. 14, the analysis of the unfavorable intermediate risk and high risk patients shows that although the biopsy Gleason score does stratify patients for difference in clinical recurrence outcomes this parameter mostly indicates men at high risk of biochemical recurrence after surgery. This is, in particular, true for the small group of men with a biopsy Gleason score 4+4.

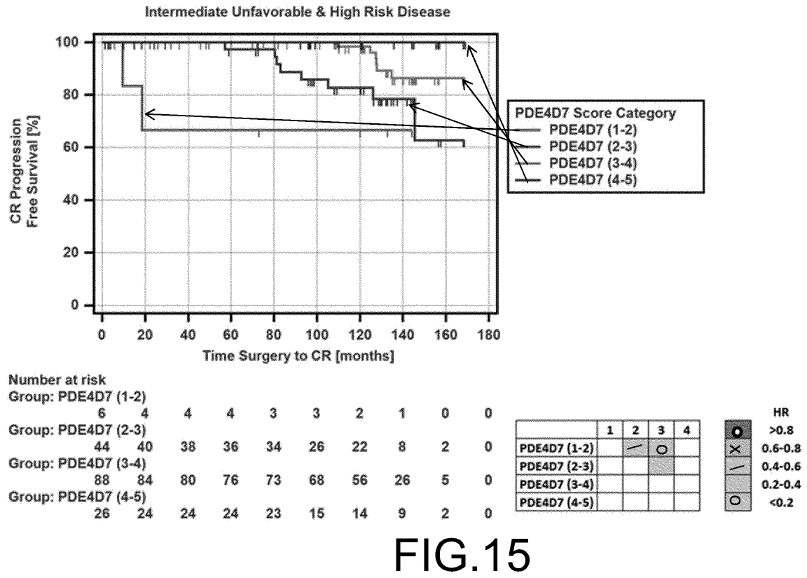

In contrast, with reference to FIG. 15, the PDE4D7 risk categories sub-stratify patients into two risk groups with highest PDE4D7 scores (3-5) with very little risk of clinical recurrence over 10 years after surgery (only 1 event in 114 patients; 0.9%). On the other hand, the events in the lowest PDE4D7 risk category (2 out of 6) occur within 20 months after surgery indicating not only a high recurrence risk in this patient group (33.3%) but also fast relapse after surgery in case a recurrence occurs.

Thus, the quantification of PDE4D7 into a risk score for patients with prostate cancer adds independent and complementary value to risk stratification of populations defined by clinical parameters. In particular, high levels of PDE4D7 expression might be able to provide extra decision power to select patients with lower risk compared to clinical information alone across all clinical risk groups. At the same time, low PDE4D7 expression might contribute to re-stratification of patients with very high risk of fast failure on endpoints like PSA relapse. Moreover, the PDE4D7 risk score determined as disclosed herein is able to sub-stratify patients into different progress-free survival risks, which was not possible by the other risk determinations.

Combination of PDE4D7 Risk Score with a Second Risk Determination

The data presented is in this specification indicate that the risk of disease progression provided by the PDE4D7 risk score offers a novel insight into prostate cancer sub-populations and thus is set to be complementary to the risks provided by current clinical practice criteria (second risk determination). It was therefore hypothesized that the combination of both risk scores by computational modelling might predict long-term disease outcomes more effectively compared to using any single score alone. To evaluate this hypothesis a sub-cohort of 449 patients (92 events; 20.5%) with complete 5-year outcome histories was selected and a logistic regression model was generated in order to predict the 5-year risk of biochemical recurrence after surgery. The modelling proved the independent predictive value of the PDE4D7 risk score (Odds ratio=0.42; 95% CI=0.28-0.63; p<1.0E−04; TABLE 3). The logit function of the regression model that was used to predict the individual 5-year progression risk (BCR) for all 449 patients was:

logit(p)=A+(B*LR)+(C*FIR)++(D*UIR)+(E*HR)+(F*PDE4D7_score), (3)

where LR=NCCN low risk, FIR=NCCN favourable intermediate risk, UIR=NCCN unfavourable intermediate risk, HR=NCCN high risk, A, B, C, D, E, and F are weights, and PDE4D7_score is the continuous PDE4D7 risk score.

In particular embodiments, A may be about (−0.5)-0.5, such as 0.16, B may be about 0.0-1.0, such as 0.59, C may be about 0.0-2.0, such as 1.07, D may be about 1.0-3.0, such as 2.03, E may be about 2.0-3.0, such as 2.52, and F may be about (−1.5)-(−0.5), such as −0.87. In calculating the regression model, a value of 1 was inserted in the logit function for the NCCN risk category into which a patient falls and a value of 0 was inserted for the other NCCN risk categories. For example, if a patient was categorized as NCCN high risk, a value of 1 was inserted in the logit function for HR and a value of 0 was inserted for each of LR, FIR and UIR.

The probability p for a patient to experience the predicted event is

p=1/(1+e^{(-logit(p)}) (4)

The probability p provides a combined prognostic risk score that is based on the PDE4D7 risk score and a second risk determination, here, an NCCN classification (in the following also NCCN & PDE4D7 risk score). The combined prognostic risk score can be classified or categorized into one of at least two risk groups. For example, there may be two risk groups, or three risk groups, or four risk groups, or more than four predefined risk groups. Each risk group covers a respective range of (non-overlapping) probabilities p. For example, a risk group may include all probabilities p from 0.0 to <0.1, another risk group from 0.1 to <0.25, another risk group from 0.25 to <0.5, and another risk group from 0.5 to <1.0.

TABLE 9 shows further details on the logistic regression model. As mentioned above, as the input variables of the model the PDE4D7 risk score was used in combination with the NCCN clinical risk categories: very low risk (VLR), low risk (LR), favorable intermediate risk (FIR), unfavorable intermediate risk (UIR), and high risk (HR) of disease progression. The group with very low clinical risk was defined as the reference group, i.e., for a patient who falls into the NCCN clinical risk category VLR the NCCN & PDE4D7 risk score is only calculated via the weight A from equation (3) (i.e., the regression coefficient for “Constant” in the table) and the weighted continuous PDE4D7 risk score F*PDE4D7_score from equation (3) (the weight F being the regression coefficient for “PDE4D7_score” in the table). The respective parameters of the logistic regression modeling are indicated in the table. The regression coefficients (weights) to build the logit(p) function are given as well as the Odds ratios, 95% confidence intervals (95% CI), and p-values. In addition, standard errors and Wald statistics are shown.

TABLE 9
Logistic regression model to predict 5-year biochemical
recurrence after surgery (449 patients).

| Coefficients and | Regression | Standard | | | | |
|---|---|---|---|---|---|---|
| Standard Errors | coefficient | Error | Wald | p value | Odds ratio | 95% CI |
| Variable | | | | | | |
| NCCN very low | — | — | — | — | — | — |
| risk (reference) | | | | | | |
| NCCN low risk | 0.59 | 0.60 | 0.99 | 3.19E−01 | 1.81 | 0.56 to 5.84 |
| NCCN favorable | 1.07 | 0.59 | 3.35 | 6.74E−02 | 2.92 | 0.93 to 9.12 |
| intermediate risk | | | | | | |
| NCCN unfavorable | 2.03 | 0.56 | 12.88 | 3.00E−04 | 7.58 | 2.51 to 22.9 |
| intermediate risk | | | | | | |
| NCCN high risk | 2.52 | 0.61 | 16.93 | <1.0E−04 | 12.37 | 3.73 to 41.0 |
| PDE4D7 score | −0.87 | 0.20 | 18.27 | <1.0E−04 | 0.42 | 0.28 to 0.65 |
| Constant | 0.16 | 0.82 | 0.04 | 8.44E−01 | — | — |

Testing of the regression model showed good calibration of expected vs. observed events in both the event and no-event groups:

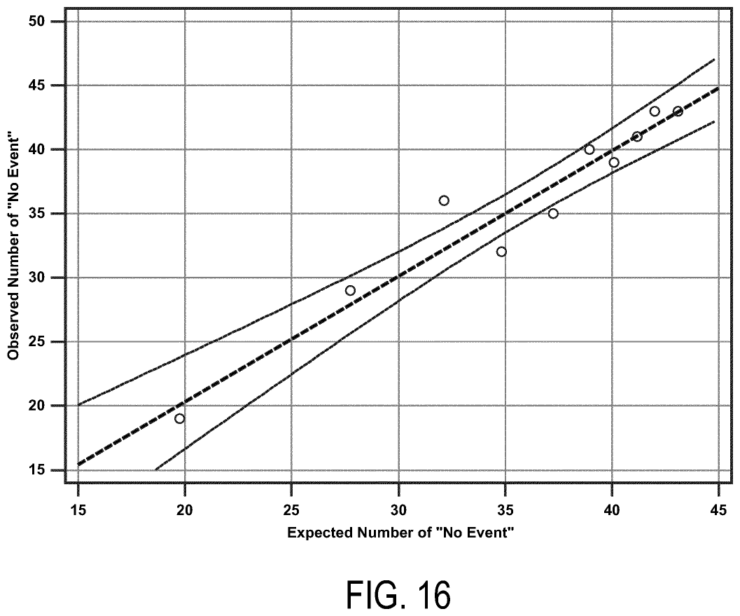

FIG. 16 shows a calibration plot of the NCCN & PDE4D7 score logistic regression model to predict 5-year biochemical relapse after surgery based on a contingency table after Hosmer-Lemeshow testing (Chi-squared 3.9; p=0.87) of the 449 patient cohort with complete 5-year follow-up. The graph shows the observed vs. the expected (i.e., logistic regression model predicted) number of patients who had no PSA relapse within 5 years after primary treatment in the ten decile sub-groups of the overall cohort. The coefficient of determination is 0.9336, the details of the equation for the plotted regression line y=Intercept+Slope*x (straight dashed line) are as follows: Intercept=0.7006 with standard error=3.3621, 95% CI=−7.0525-8.4537, t=0.2084 and p=0.8401; Slope=0.9804 with standard error=0.09243, 95% CI=0.7672-1.1935, t=10.6072 and p<0.0001. The curved dashed lines indicate the 95% confidence interval.

FIG. 17 shows a calibration plot of the NCCN & PDE4D7 score logistic regression model to predict 5-year biochemical relapse after surgery based on a contingency table after Hosmer-Lemeshow testing (Chi-squared 3.9; p=0.87) of the 449 patient cohort with complete 5-year follow-up. The graph shows the observed vs. the expected (i.e., logistic regression model predicted) number of patients who had a PSA relapse within 5 years after primary treatment in the ten decile sub-groups of the overall cohort. The coefficient of determination is 0.9291, the details of the equation for the plotted regression line y=Intercept+Slope*x (straight dashed line) are as follows: Intercept=0.2083 with standard error=1.0895, 95% CI=−2.3041-2.7207, t=0.1912 and p=0.8532; Slope=0.9774 with standard error=0.09544, 95% CI=0.7573-1.1975, t=10.2405 and p<0.0001. The curved dashed lines indicate the 95% confidence interval.

A ROC (Receiver Operating Characteristic) analysis was then performed to calculate the 2-, 5-, and 10-year AUCs (Area under the ROC Curve) as 0.779, 0.749, and 0.748, respectively:

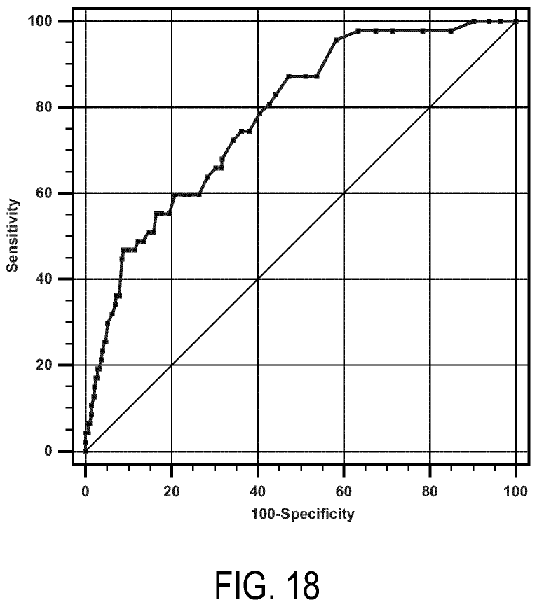

FIG. 18 shows a ROC analysis of 2-year biochemical relapse after surgery (BCR) of the NCCN & PDE4D7 score logistic regression model in a 449 patient cohort with complete 5-year follow-up. The graph shows the ROC curve of the false-positives (sensitivity) vs. the false-negatives (specificity) plot. The statistical details of the ROC analysis are as follows: Positive group=47 (10.47%), negative group 402 (89.53%), disease prevalence=10.5%, AUC=0.779, standard error=0.0334, 95% CI=0.738-0.817, z statistic=8.368, p (area=0.5)<0.0001.

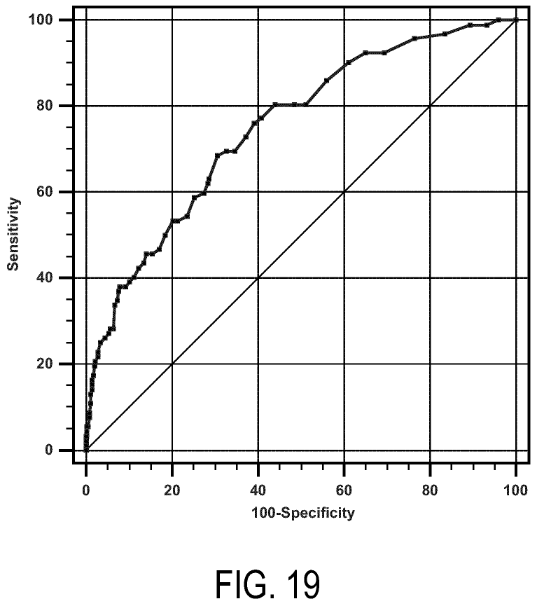

FIG. 19 shows a ROC analysis of 5-year biochemical relapse after surgery (BCR) of the NCCN & PDE4D7 score logistic regression model in the 449 patient cohort with complete 5-year follow-up. The graph shows the ROC curve of the false-positives (sensitivity) vs. the false-negatives (specificity) plot. The statistical details of the ROC analysis are as follows: Positive group=92 (20.49%), negative group 357 (79.51%), disease prevalence=unknown, AUC=0.749, standard error=0.0284, 95% CI=0.706-0.788, z statistic=8.739, p (area=0.5)<0.0001.

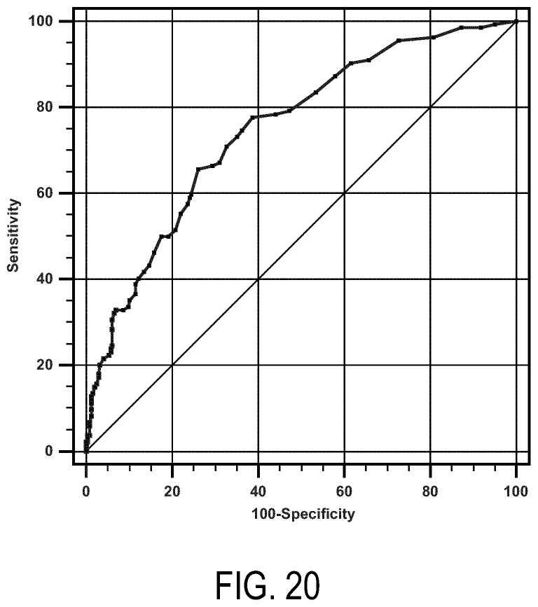

FIG. 20 shows a ROC analysis of 10-year biochemical relapse after surgery (BCR) of the NCCN & PDE4D7 score logistic regression model in a 379 patient cohort with complete 10-year follow-up. The graph shows the ROC curve of the false-positives (sensitivity) vs. the false-negatives (specificity) plot. The statistical details of the ROC analysis are as follows: Positive group=134 (35.36%), negative group 245 (64.64%), disease prevalence=unknown, AUC=0.748, standard error=0.0259, 95% CI=0.701-0.791, z statistic=9.549, p (area=0.5)<0.0001.

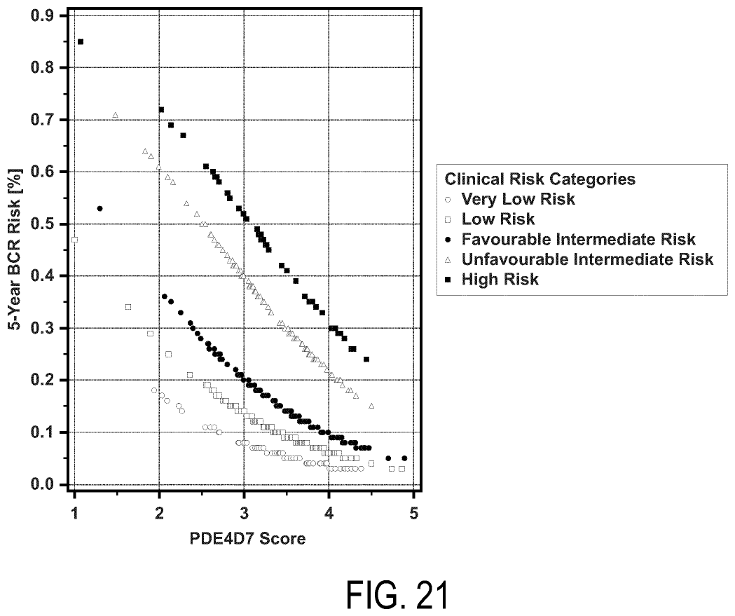

As shown in FIG. 21, a predicted risk analysis per NCCN clinical risk category as a function of the PDE4D7 risk score revealed a heterogeneous 5-year progression risk (BCR) distribution even within the lowest NCCN clinical risk groups. In the figure, the 5-year predicted probability p is plotted against the PDE4D7 risk scores for each individual NCCN risk category.

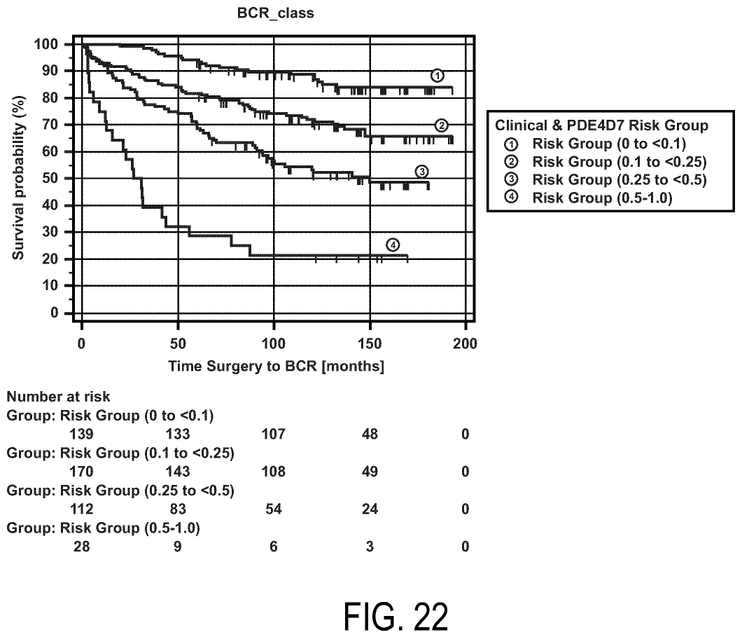

Based on the predicted risk per patient, four risk groups were defined with either low likelihood of 5-year (<10%) or 10-year (<15%) BCR after primary treatment (risk group 0 to <0.1) in FIG. 22) or with a very considerable 5-year post-surgical risk (>700%) of PSA failure (risk group (0.5 to 1.0) in FIG. 22) with a median risk to event of only 27.1 months. The median risk for risk group (0.25 to <0.5) was 149.7 months while the median progression free survival was not reached for the risk groups (0 to <0.1) and (0.1 to <0.25). When using risk group (0 to <0.1) as a reference in the Kaplan-Meier analysis the hazard ratios for risk group (0.1 to <0.25), (0.25 to <0.5), and (0.5 to 1.0) were 2.2 (9500 CI 1.7-3.5), 4.3 (95% CI 2.8-6.6), and 11.5 (95% CI 4.6-28.8), respectively (FIG. 22; TABLE 10).

TABLE 10
Hazard ratio table for the NCCN & PDE4D7 score logistic regression model in Kaplan Meier
survival analysis of the clinical endpoint biochemical recurrence free survival

| | | NCCN & | NCCN & | NCCN & | NCCN & |
|---|---|---|---|---|---|
| | HR/ | PDE4D7 Risk | PDE4D7 Risk | PDE4D7 Risk | PDE4D7 Risk |
| Risk Group | 95% CI | Group (1) | Group (2) | Group (3) | Group (4) |
| NCCN & | HR | — | 2.4 | 4.3 | 11.5 |
| PDE4D7 Risk | 95% CI | | 1.7-3.5 | 2.8-6.6 | 4.6-28.8 |
| Group (1) | | | | | |
| NCCN & | HR | 0.42 | — | 1.8 | 4.8 |
| PDE4D7 Risk | 95% CI | 0.29-0.6 | | 1.6-2.7 | 1.9-11.9 |
| Group (2) | | | | | |
| NCCN & | HR | 0.23 | 0.56 | — | 2.7 |
| PDE4D7 Risk | 95% CI | 0.15-0.36 | 0.37-0.86 | | 1.1-6.9 |
| Group (3) | | | | | |
| NCCN & | HR | 0.09 | 0.20 | 0.37 | — |
| PDE4D7 Risk | 95% CI | 0.03 to 0.21 | 0.08 to 0.52 | 0.14-0.95 | |
| Group (4) | | | | | |

### Discussion

In order to assess an individual risk on a per patient basis, a regression model based on the NCCN risk group and the PDE4D7 risk score was developed. The regression model showed that the PDE4D7 risk score defined widely overlapping distributions of individual risks in the contemporary NCCN risk categories. Irrespective of the NCCN risk group that was determined for an individual patient, a higher PDE4D7 risk score defined a lower risk of progression, while a lower PDE4D7 risk score indicated a higher risk of progressive disease for a given patient.

Very recently, the long-term results of the active surveillance cohort within the Göteborg randomized prostate cancer screening trial were published. These indicate that men with a clinically low risk disease may have a considerable risk to experience a progressive disease under a deferred treatment regime. Therefore, the authors questioned whether men other than those with very low risk disease would be eligible for expectant management strategies. The recent publication of the 10-year outcomes of the ProtecT study indicates similar conclusions in the active monitoring arm of the trial. Although there is some debate about the validity of these results to contemporary practice they may suggest that only patients with the very lowest risk are safe of any progression during deferred treatment management. While the use of clinical criteria allow the selection of such a very low risk, but small, patient cohort (e.g., 62 out of 449 patients in our cohort; 13.8%), the addition of molecular markers is expected by the inventors to enlarge this very low risk patient group (e.g., 122 out of 449 in our cohort when the PDE4D7 risk score is combined with the NCC risk category, corresponding to 27.2%).

As used in this specification and in the appended claims, the singular forms of “a” and “an” also include the respective plurals unless the context clearly dictates otherwise.

The terms “about” and “approximately” denote an interval of accuracy that a person skilled in the art will understand to still ensure the technical effect of the feature in question. The term typically indicates a deviation from the indicated numerical value of 20%, or 15%, or 10%, or 5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which consists of these embodiments only.

Furthermore, the terms “first,” “second,” “third” or “(a),” “(b),” “(c),” “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first,” “second,” “third” or “(a),” “(b),” “(c),” “(d)” etc. relate to steps of a method or use there is no time or time interval coherence between the steps, i.e., the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, proteins, bacteria, vectors, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The attached Sequence Listing, entitled 2014PF01672_Sequence Listing_ST25 is incorporated herein by reference, in its entirety.

1. A method of risk stratification comprising:
determining a normalized gene expression profile for a single marker gene consisting of phosphodiesterase 4D variant 7 (PDE4D7), with respect to a set of reference gene(s) selected from the group consisting of: Homo sapiens hypoxanthine phosphoribosyltransferase 1 (HPRT1), Tubulin-Alpha-1b (TUBA1B), Homo sapiens pumilio RNA-Binding Family Member (PUM1) and Homo sapiens TATA box binding protein (TBP), and combinations thereof;
determining a prognostic risk score with a scoring function, based on the normalized gene expression profile, the scoring function having been derived from gene expression profiles for biological samples taken from subjects that have been monitored for prostate cancer; and
determining a combined prognostic risk score based on the prognostic risk score and a second risk determination being a National Comprehensive Cancer Network classification.

2. (canceled)

3. (canceled)

4. The method of claim 1, further characterized by the combined prognostic risk score being determined with a regression function derived from subjects that have been monitored for prostate cancer.

5. The method of claim 1, further characterized by the at least one reference gene including at least two of, or at least three of, or all of HPRT1, TUBA1B, PUM1, and TBP.

6. The method of claim 1, further characterized by the prognostic risk score being based on the normalized gene expression profile that includes the expression level for PDE4D7 as the only marker gene.

7. The method of claim 6, further characterized by the determining of the gene expression profile comprising performing RT-qPCR on RNA extracted from a biological sample.

8. The method of claim 7, further characterized by the determining of the gene expression profile including determining a Cq value for PDE4D7 and each of the at least one reference gene and by the determining a prognostic risk score including normalizing the PDE4D7 value using the value of each of the reference genes in the set and computing the risk score as a linear function of the normalized score.

9. The method of claim 8, further characterized by categorizing a subject into one of a predefined set of at least two or at least three risk groups, based on the combined prognostic risk score.

10. The method of claim 9, further comprising at least one of:
proposing a therapy for a subject based on the assigned risk group, at least two of the risk groups being associated with different therapies;
computing a disease progression risk prediction of the subject before or after prostate surgery; and
computing a therapy response prediction for the subject before or after prostate surgery.

11. The method of claim 10, further characterized by the proposed therapy being selected from the group consisting of:
a) at least a partial prostatectomy;
b) an active therapy selected from radiation treatment, hormone therapy, chemotherapy, and a combination thereof,
c) observation without performing a) or b).

12. The method of claim 11, further characterized by the proposed therapy based on the assigned risk group being different from a proposed therapy based only on the second risk determination.

13. A computer program product storing instructions which, when executed by a computer, performs the method of claim 10.

14. A diagnostic kit, the kit comprising:
at least one primer and/or probe for determining the expression level of phosphodiesterase 4D variant 7 (PDE4D7);
at least one primer and/or probe for determining the gene expression level of at least one reference gene selected from the group consisting of: Homo sapiens hypoxanthine phosphoribosyltransferase 1 (HPRT1), Tubulin-Alpha-1b (TUBA1B) Homo sapiens pumilio RNA-Binding Family Member (PUM1) and Homo sapiens TATA box binding protein (TBP), and combinations thereof, and
instructions for computing a risk score based on the determined expression levels, the instructions being stored on a computer program product which, when executed by a computer, perform a method comprising:
determining a normalized gene expression profile for the phosphodiesterase 4D variant 7 (PDE4D7), with respect to the at least one reference gene;
determining a prognostic risk score with a scoring function, based on the normalized gene expression profile; and
determining a combined prognostic risk score based on the prognostic risk score and a second risk determination being a National Comprehensive Cancer Network classification.

15. Use of a gene expression profile for risk stratification, comprising:
determining a gene expression profile of a biological sample obtained from a subject, wherein the gene expression profile is an expression level for phosphodiesterase 4D variant 7 (PDE4D7) normalized with respect to a set of reference gene(s) selected from the group consisting of: Homo sapiens hypoxanthine phosphoribosyltransferase 1 (HPRT1), Tubulin-Alpha-1b (TUBA1B) Homo sapiens pumilio RNA-Binding Family Member (PUM1) and Homo sapiens TATA box binding protein (TBP), and combinations thereof, and
determining a prognostic risk score for the subject based on the gene expression profile with a scoring function that is derived from gene expression profiles for biological samples taken from subjects that have been monitored for prostate cancer; and
determining a combined prognostic risk score based on the prognostic risk score and a second risk determination being a National Comprehensive Cancer Network classification.

16. A computer program product comprising a non-transitory recording medium storing instructions, which when executed on a computer, cause the computer to perform a method comprising:
computing a normalized gene expression profile for phosphodiesterase 4D variant 7 (PDE4D7), with respect to a set of reference gene(s) selected from the group consisting of: Homo sapiens hypoxanthine phosphoribosyltransferase 1 (HPRT1), Tubulin-Alpha-1b (TUBA1B) Homo sapiens pumilio RNA-Binding Family Member (PUM1) and Homo sapiens TATA box binding protein (TBP), and combinations thereof,
computing a prognostic risk score for the subject based on the gene expression profile with a scoring function that is optionally derived from gene expression profiles for biological samples taken from subjects that have been monitored for prostate cancer; and
computing a combined prognostic risk score based on the prognostic risk score and a second risk determination being a National Comprehensive Cancer Network classification.
